(19) United States
(12) Patent Application Publication
(10) Pub. No.: US 2026/0116941 A1
(43) Pub. Date: Apr. 30, 2026

(54) MODIFIED MAMMALIAN VESICLES AND COMPOSITIONS AND METHODS RELATED THERETO

(71) Applicant: WAKE FOREST UNIVERSITY HEALTH SCIENCES, Winston-Salem, NC (US)

(72) Inventors: Kounosuke WATABE, Summerfield, NC (US); Kerui WU, Apex, NC (US)

(21) Appl. No.: 19/471,869

(22) PCT Filed: Apr. 10, 2024
(86) PCT No.: PCT/US2024/023802

§ 371 Date: Oct. 2, 2025

Related U.S. Application Data
(60) Provisional application No. 63/495,171, filed on Apr. 10, 2023.

(51) Int. Cl. C07K 14/55; A61K 35/15; A61K 39/00; A61P 35/00; C07K 14/47; C07K 14/705; C07K 14/74

(52) U.S. Cl. CPC C07K 14/55; A61K 35/15; A61K 39/00114; A61P 35/00; C07K 14/4748; C07K 14/70539; C07K 14/70578; A61K 2039/55533; A61K 2039/605; C07K 2319/00

(57) ABSTRACT

This invention relates engineered extracellular vesicles (EVs) comprising a membrane-bound interleukin-2 (IL-2) molecule and/or functional fragment thereof, a major histocompatibility (MHC) molecule and/or functional fragment thereof, and one or more costimulatory molecule, each expressed on the surface membrane of the EV. The invention further relates to compositions, cells and kits comprising the same, and methods of using and making the same.

### PRIORITY STATEMENT

This application claims the benefit of U.S. Provisional Application Ser. No. 63/495,171, filed Apr. 10, 2023, the entire contents of which are incorporated by reference herein.

### STATEMENT OF GOVERNMENT SUPPORT

This invention was made with government support under Grant Numbers CA173499, CA185650 and CA205067 awarded by the National Institutes of Health, and Grant Number W81XWH2110075 awarded by the Department of Defense. The government has certain rights in the invention.

### FIELD OF THE INVENTION

This invention relates engineered extracellular vesicles (EVs) comprising a membrane-bound interleukin-2 (IL-2) molecule and/or functional fragment thereof, a major histocompatibility (MHC) molecule and/or functional fragment thereof, and one or more costimulatory molecule, each expressed on the surface membrane of the EV. The invention further relates to compositions, cells and kits comprising the same, and methods of using and making the same.

### BACKGROUND OF THE INVENTION

Several new immunotherapeutic agents have changed the treatment strategies and improved the outcome of certain cancers in clinical practice. Immune checkpoint inhibitors (ICIs) have shown dramatic efficacy in certain patient populations of lung cancer, melanoma, and leukemia (Kim and Park 2019 Arch Pharm Res 42:567-581). However, the majority of patients still presented resistance to ICI-based therapies, and even patients with initial responses developed acquired resistance and relapse during the follow-up treatment (Jenkins, Barbie and Flaherty 2018 Br J Cancer 118:9-16). The possible causes of the resistance include low mutation burden (Egelston et al. 2019 J. Immunology 202), decreased infiltration of APCs (Gabrilovich et al. 1997 Clinical Cancer Research 3:483-490; Gervais et al. 2005 Breast Cancer Research 7:R326-335), and failure of T cells activation (Dadmarz et al. 1995 Cancer Immunol Immunother 40:1-9; Luo et al. 2018 Nat Commun 9:248).

Compared to other types of solid tumors such as lung cancer, melanoma, and colorectal cancer, breast cancer has been shown to be more resistant to immunotherapies (Adams et al. 2019 Annals of Oncology ESMO 30:397-404; Gatti-Mays et al 2019 NPJ Breast Cancer 5:37). One possible cause could be the lower tumor mutation burden compared to other types of cancers (Castle et al. 2019 Breast cancer research 8:101). There are fewer neoantigens for generating tumor-reactive immune cells in breast cancer cells. Furthermore, breast cancer patients often have impaired APCs with decreased antigen presentation, possibly through secretion of soluble factors by breast cancer cells, which have a direct negative effect on APCs (Lenahan and Avigan 2006 Breast cancer research 8:101).

The present invention overcomes previous shortcomings in the art by providing engineered extracellular vesicles for active immunotherapy, and methods of making and using the same.

### SUMMARY OF THE INVENTION

One aspect of the invention provides an isolated nonnucleated extracellular vesicle (EV) comprising a surface membrane and a fusion protein comprising an interleukin-2 (IL-2) molecule and/or functional fragment thereof, a major histocompatibility (MHC) molecule and/or functional fragment thereof (e.g., MHC-I; e.g., MHC-II), and one or more costimulatory molecule, each expressed on the surface membrane of the EV; wherein the MHC molecule and/or functional fragment thereof presents an exogenously introduced antigen.

Another aspect of the invention provides a composition comprising the isolated EV of the present invention, further comprising a pharmaceutically acceptable carrier, diluent, and/or adjuvant.

Another aspect of the invention provides a method of producing an immune response to a disorder in a subject, comprising administering to the subject an effective amount of the isolated EV and/or the composition of the present invention. Also provided is the use of the isolated EV and/or the composition of the present invention for producing an immune response to a disorder in a subject, or for the preparation of a medicament for producing an immune response to a disorder in a subject.

Another aspect of the invention provides a method of protecting a subject from the effects of a disorder, comprising administering to the subject an effective amount of the isolated EV and/or the composition of the present invention. Also provided is the use of the isolated EV and/or the composition of the present invention for protecting a subject from the effects of a disorder, or for the preparation of a medicament for protecting a subject from the effects of a disorder.

Another aspect of the invention provides a method of treating a disorder in a subject, comprising administering to the subject an effective amount of the isolated EV and/or the composition of the present invention. Also provided is the use of the isolated EV and/or the composition of the present invention for treating a disorder in a subject, or for the preparation of a medicament for treating a disorder in a subject.

In some embodiments, the disorder is cancer.

In some embodiments, the cancer is breast cancer (e.g., BRCA⁺ breast cancer, HER breast cancer, progesterone receptor (PR)⁺ breast cancer, triple-negative (BRCA⁻/HER⁻/PR⁻) breast cancer, lobular breast carcinoma, ductal breast carcinoma, breast adenocarcinoma, metastatic breast cancer).

Another aspect of the invention provides a method of producing an immune response to a cancer in a subject, comprising administering to the subject an effective amount of the isolated EV and/or the composition of the present invention. Also provided is the use of the isolated EV and/or the composition of the present invention for producing an immune response to a cancer in a subject, or for the preparation of a medicament for producing an immune response to a cancer in a subject.

Another aspect of the invention provides a method of preventing a disorder associated with a cancer in a subject, comprising administering to the subject an effective amount of the isolated EV and/or the composition of the present invention. Also provided is the use of the isolated EV and/or the composition of the present invention for preventing a disorder associated with a cancer in a subject, or for the preparation of a medicament for preventing a disorder associated with a cancer in a subject.

Another aspect of the invention provides a method of protecting a subject from the effects of a cancer, comprising administering to the subject an effective amount of the isolated EV and/or the composition of the present invention. Also provided is the use of the isolated EV and/or the composition of the present invention for protecting a subject from the effects of a cancer, or for the preparation of a medicament for protecting a subject from the effects of a cancer.

Another aspect of the invention provides a method of treating to a cancer in a subject, comprising administering to the subject an effective amount of the isolated EV and/or the composition of the present invention.

Another aspect of the invention provides a method of producing a nonnucleated extracellular vesicle comprising a surface membrane and comprising an interleukin-2 (IL-2) molecule and/or functional fragment thereof, a major histocompatibility (MHC) molecule and/or functional fragment thereof, and one or more costimulatory molecule, each expressed on the surface membrane of the EV, wherein the MHC molecule and/or functional fragment thereof presents an exogenously introduced antigen (e.g., the isolated EV of the present invention), the method comprising: (a) delivering to a culture of one or more antigen presenting cell that expresses major histocompatibility molecules I and II and which secretes nonnucleated EVs derived from the surface membrane of the APC, a IL-2 molecule and/or functional fragment thereof formulated to be expressed on the surface membrane of the APC, thereby producing an APC expressing the IL-2 molecule and/or functional fragment thereof on the surface membrane of the APC; (b) contacting the APC of step (a) with one or more innate immune activator, thereby inducing expression of one or more costimulatory molecules on the surface membrane of the APC; (c) introducing to the APC of step (b) a source of exogenous antigen, thereby producing an APC expressing MHC-I and MHC-II, one or more costimulatory molecule and the IL-2 molecule and/or functional fragment thereof, on the surface membrane of the APC and on the surface membrane of the EVs secreted by the APC, wherein the MHC-I and/or MHC-II expressed on the surface membrane of the APC and the EVs presents the introduced exogenous antigen, thereby producing a nonnucleated EV comprising a surface membrane and expressing MHC-I and MHC-II, one or more costimulatory molecule and the IL-2 molecule and/or functional fragment thereof, on the surface membrane of the EV, wherein the MHC-I and/or MHC-II expressed on the surface membrane of the EV presents the introduced exogenous antigen.

Another aspect of the invention provides a method of treating a cancer (e.g., breast cancer) in a subject in need thereof, comprising: (a) retrieving a sample from the subject, wherein the sample comprises an APC of the subject that expresses major histocompatibility molecules I and II and which secretes nonnucleated EVs derived from the surface membrane of the APC; (b) isolating one or more APC from the sample; (c) delivering to a culture comprising one or more APC isolated from the sample, a IL-2 molecule and/or functional fragment thereof formulated to be expressed on the surface membrane of the APC; (d) contacting the APC of step (c) with one or more innate immune activator, thereby inducing expression of one or more costimulatory molecules on the surface membrane of the APC; (e) introducing to the APC of step (d) a source of cancer antigen from the cancer of the subject, thereby producing an APC expressing MHC-I and MHC-II, one or more costimulatory molecule and the IL-2 molecule and/or functional fragment thereof, on the surface membrane of the APC and on the surface membrane of the EVs secreted by the APC, wherein the MHC-I and/or MHC-II expressed on the surface membrane of the APC and the EVs presents the introduced cancer antigen from the cancer of the subject, (f) isolating the EVs secreted from the APC of step (e); and (g) administering to the subject an effective amount of the isolated EVs and/or a composition comprising the same, thereby treating the cancer in the subject in need thereof.

Also provided is the use of the isolated EV and/or the composition of the present invention for treating cancer (e.g., breast cancer) in a subject in need thereof, or for the preparation of a medicament for treating cancer (e.g., breast cancer).

### BRIEF DESCRIPTION OF THE DRAWINGS

FIG. 1 panels A-F show images of schematics, fluorescence microscopy and data plots relating to engineering of membrane-bound IL2 and induction of co-stimulatory factors on the surface of p13nsEV. (FIG. 1 panel A) The structure of lentiviral plasmid for ectopic expression of IL2-MFG-E8 fusion protein on the membrane of p13nsEV. (FIG. 1 panel B) The schematic diagram illustrating the proposed approach to anchor IL2 on the surface of p13nsEV so that it will bind to IL2 receptor on T cells. (FIG. 1 panel C) The membrane fraction of p13nsEV and DCs before and after IL2 expression was isolated, and the expression of IL2 and HSP70 was examined by Western Blot. (FIG. 1 panel D) Flow cytometry of IL2 expression on the surface of p13nsEV with or without surface IL2 expression. (FIG. 1 panels E and F) Vybrant DiD-labeled T lymphocyte was co-cultured with p13nsEV/PlamGFP and IL2-p13nsEV/PalmGFP. After 12 hrs, the interaction between T cells and sEVs was examined and quantified by fluorescent microscopy (FIG. 1 panel E) and (FIG. 1 panel F) flow cytometry. The comparison was performed using unpaired student t test. *P<0.05, ***P<0.001, ****P<0.0001. Scale bar indicates 100 μm.

FIG. 2 panels A-G show images of data plots and fluorescence microscopy relating to IL2-ep13nsEV induction of increased immunity of T cells against cancer cells. (FIG. 2 panel A) p13nsEV or IL2-p13nsEV was labeled using the ExoGlow™-Vivo EV Labeling Kit and injected into tail base of mice. After 6 hours, the organ distributions of the vesicle were examined and quantified by IVIS Spectrum. Two sailed unpaired t tests were performed to compare the signal strength in different organs. (FIG. 2 panel B) Western blot analysis was performed for the T cells that were treated with either p13nsEV, or IL2-p13nsEV with or without IL-2Rα antagonist to examine the activation of IL2 receptor by IL2-p13nsEV. (FIG. 2 panel C) The DCs were treated with different combinations of cytokines for maturation. Then 4-1BBL and CD40L positive DCs were examined by flow cytometry, and mean fluorescence intensity was recorded and compared by two tailed unpaired t test. (FIG. 2 panel D) The DCs were treated with different combinations of cytokines for maturation. The p13nsEV from DCs were isolated by ultracentrifuge. The 4-1BBL positive and CD40L positive p13nsEV were examined by flow cytometry, and mean fluorescence intensity was recorded and compared by two tailed unpaired t tests. (FIG. 2 panel E) DCs with or without IL2-MFG-E8, LPS/C-diGMPT enhanced co-stimulatory factors were pulsed with 250 μg/ml OVA and p13nsEV was purified. CD8 T cells from OT-I mice cells were then co-cultured with different types of p13nsEV or pulsed DC for 5 days. Flow cytometry was performed to analyze the activated IFN-γ+ CD8 T cells. Isotype IgG control was used to determine the baseline signal. Two tailed unpaired t tests were performed to compare the populations. (FIG. 2 panel F) The CD8 T cells from (FIG. 2 panel E) were co-cultured with B16-OVA cells expressing GFP at 10:1 ratio (T cell:Tumor cell). Flow cytometry was performed to quantify the dead cancer cells by Zombie Aqua™ dead cell labelling dye among GFP+ cells. Two tailed unpaired t tests were performed to compare the populations. (FIG. 2 panel G) The Vybrant DiD-labeled p13nsEV was injected into tail base of mice, and the lymph nodes were taken out after 6 hours, and the sections were stained for lymphocyte markers and examined by microscope. Scale bar indicates 100 μm. *P<0.05, **P<0.01, ***P<0.001, ****P<0.0001.

FIG. 3 panels A-I show images of schematics and data plots relating to the effects of engineered IL2-ep13nsEV on tumor growth in syngeneic mice models. (FIG. 3 panel A) Schematic diagram of experimental procedure. Tumor cell lysate from EO771 was loaded into mBMDC with or without IL2-MFG-E8 expression, followed by DC maturation and STING agonist Cyclic diGMP (c-diGMP) treatment. The sEV was isolated from the DC and used for the treatment of EO771 tumor bearing C57BL/6J mice at five different time points, starting from one week before the tumor cell inoculation as indicated. PBS and DC pulsed with tumor lysate were used in the control groups. (FIG. 3 panel B) Tumor growth on mammary fat pad was monitored by measuring the tumor size by a caliper. Two tailed unpaired t test was performed to compare the tumor sizes at different time points. (FIG. 3 panel C) The tumor weight at the end point (Day 25) is shown for the different treatment groups. Two tailed unpaired t test was performed to compare the tumor weight. (FIG. 3 panel D) The tumors were dissociated, and CD4+ and CD8+ TIL among CD3+ cells were measured by flow cytometry for each group and two tailed unpaired t test was performed to compare the percentage of TIL. (FIG. 3 panel E) The CD3−, NK1.1+ NK cells in the tumor were examined by flow cytometry. The percentage of NK cells was compared by two-tailed unpaired t-test between groups. (FIG. 3 panel F) The CD25+ and Foxp3+ regulatory T (Tregs) cells among CD3+/CD4+ tumors were examined by flow cytometry. The percentage of Tregs cells was compared by two tailed unpaired t-test between different groups. (FIG. 3 panel G) The CD4+ and CD8+ cells among IFN-γγ+/CD3+ in dissociated splenocytes were examined by flow cytometry. The percentage of cells was compared between different groups by two tailed unpaired t test. (FIG. 3 panel H) APC cells were depleted in CD11c-DTR mice by administration of diphtheria toxin, anti-CD20 and Clophosome 24 hours before IL2-ep13nsEV treatment. The mice in APC+ group received injection of IgG2c isotype control and empty liposome. 50 μg of IL2-ep13nsEV was given to mice 5 times as indicated in 3A. Tumor growth on mammary fat pad was monitored by measuring the tumor size by a caliper. Two tailed unpaired t test was performed to compare the tumor sizes. (FIG. 3 panel I) The tumors were dissociated, and CD4+ and CD8+ TIL among CD3+ cells were measured by flow cytometry for each group and two tailed unpaired t test was performed to compare the percentage of TIL. *P<0.05, **P<0.01, ***P<0.001, *P<0.0001.

FIG. 4 panels A-M show images of schematics, photographs, data plots and fluorescence microscopy relating to the effects of combination treatment of engineered IL2-ep13nsEV and ICI on breast tumor in syngeneic mice models. (FIG. 4 panel A) Schematic diagram of experimental procedure. Tumor cell lysate from 4T1 was loaded into mBMDC with IL2-MFG-E8 expression, followed by DC maturation and STING agonist c-diGMP treatment. The IL2-ep13nsEV was isolated from the DC and used for the treatment of tumor bearing BALB/C mice at five different time points, starting from one week before the tumor cell inoculation as indicated. PBS was used in the control group. (FIG. 4 panel B) Tumor growth on mammary fat pad was monitored by measuring the tumor size by a caliper. Two tailed unpaired t test was performed to compare the tumor sizes. (FIG. 4 panel C) The tumor weight at the end point (Day 28) is shown for the two different treatment groups. Two tailed unpaired t test was performed to compare the tumor weight. (FIG. 4 panel D) The tumors were dissociated, and CD4+ and CD8+ TIL among CD3+ cells were measured by flow cytometry for each group and two tailed unpaired t test was performed to compare the percentage of TIL. (FIG. 4 panel E) The IFN-γγ+/CD4+ and IFN-γγ+/CD8+ among CD3+ cells in dissociated splenocytes were examined by flow cytometry. The percentage of cells was compared by two tailed unpaired t test. (FIG. 4 panel F) Schematic diagram of experimental procedure. Tumor cell lysate from 4T1 was loaded into mBMDC with IL2-MFG-E8 expression, followed by DC maturation and STING agonist c-diGMP treatment. The IL2-ep13nsEV was isolated from the DC and used for the treatment of tumor bearing BALB/C mice at five different time points starting from one week before the tumor cell inoculation as indicated. (FIG. 4 panel G) One week after the tumor cell inoculation, one group of mice was treated with immune checkpoint inhibitor, anti-PD1, every four days for four times. (FIG. 4 panel H) Tumor growth on mammary fat pad was monitored by measuring the tumor size by a caliper. Two tailed unpaired t test was performed to compare the tumor sizes at different time points. Purified sEV from DC without tumor lysate pulsed was used as vesicle control of IL2-ep13nsEV4T1. (FIG. 4 panel I) The tumor weight at the end point (Day 28) for the four treatment groups is shown. Two tailed unpaired t test was performed to compare the tumor weight. (FIG. 4 panels J and K) The intratumoral CD8+ and CD4+ cells from different treatment groups were stained and counted. Two tailed unpaired t test was performed to compare the amount of TIL. Scale bar indicates 50 μm. (FIG. 4 panels L-M) The IFN-γγ+/CD4+ (FIG. 4 panel L) and IFN-γγ+/CD8+ (FIG. 4 panel M) cells among CD3+ cells in dissociated splenocytes were examined by flow cytometry. The percentage of cells was compared among different groups by two tailed unpaired t test. n.s. P≥0.05, *P<0.05, **P<0.01, ***P<0.001, ****P<0.0001.

FIG. 5 panels A-I show images of schematics, data plots and tissue histology relating to the effects of combination therapy of engineered IL2-ep13nsEV and ICI on breast tumor growth in humanized PDX mice. (FIG. 5 panel A) Schematic diagram of experimental procedure. Tumor cell lysate from PDX was loaded into human monocyte-derived DC with IL2-MFG-E8 expression, followed by DC maturation and STING agonist c-diGMP treatment. The IL2-ep13nsEV was isolated from the DC and used as active immunotherapy to treat humanized PDX mice at five different time points, starting from three weeks after the tumor cell inoculation as indicated. Five weeks after the tumor cell inoculation, one group of mice were treated with immune checkpoint inhibitor, anti-PD1, once every two weeks for four times. (FIG. 5 panel B) Tumor growth on mammary fat pad was monitored by measuring the tumor size by a caliper. Two tailed unpaired t test was performed to compare the tumor sizes at different time points. The sEV purified from DC pulsed with lysate of health mammary fat pad was used as vesicle control of IL2-ep13nsEV3887. Isotype IgG1 was used as the control of anti-PD1 treatment. (FIG. 5 panel C) The tumor weight at the end point for the four treatment groups is measured and compared by two tailed unpaired t test. (FIG. 5 panel D) The tumor incidence of four groups was compared by the Chi-square test. (FIG. 5 panels E and F) The tumors were dissociated and hCD45+/hCD3+, CD4+ (FIG. 5 panel E) and CD8+ (FIG. 5 panel F) TIL were measured by flow cytometry for each group and compared by unpaired t test. (FIG. 5 panels G and H) The hCD45+/hCD3+/IFN-γγ+, CD4+ (FIG. 5 panel G) and CD8+ (FIG. 5 panel H) cells in dissociated splenocytes were examined by flow cytometry. The percentage of cells was compared among different groups by two tailed unpaired t test. (FIG. 5 panel I) The intratumoral CD4 and CD8 TIL were examined by IHC and compared by two tailed unpaired t test. Scale bar indicates 50 μm. n.s. P≥0.05, *P<0.05, **P<0.01, ***P<0.001, ****P<0.0001.

FIG. 6 panels A-K show images of schematics, tissue histology, and data plots relating to the effects of IL2-ep13nsEV on lung metastasis after surgery. (FIG. 6 panel A) Schematic diagram of testing IL2-ep13nsEV in the in vivo breast carcinoma recurrence model. 4T1 cells were injected into mammary fat pads of mice. After one week, surgery was performed to remove the primary tumor. The extracted tumor tissue was used to prepare the tumor lysate and loaded into IL2-MFG-E8 engineered mBMDC. After inducing maturation and STING agonist C-diGMP treatment, IL2-ep13nsEV was purified from the DC and they were administered to the mice after the surgical removal of tumor. The IL2-ep13nsEV was given once a week for four times starting from 3 days after primary tumor removal. At day 35 after the surgery, the lungs were removed and examined for metastases. (FIG. 6 panel B) The sections of lungs were stained by H&E. (FIG. 6 panel C) The metastatic nodules were counted and compared between the two groups by two tailed unpaired t test. (FIG. 6 panel D) Same treatments were given to another pair of mice to test the survival after treatment. Log-rank (Mantel-Cox) test was performed to compare the two groups. (FIG. 6 panel E) The CD3+, CD4+ (left panel) and CD8+ (right panel) cells in the blood were examined by flow cytometry. The percentage was quantified and compared between the two groups by unpaired t test. (FIG. 6 panels F and G) The sections of lungs were stained for CD4 and CD8 cells. The numbers of CD4 (left panel) and CD8 (right panel) cells were also quantified, and compared between the two groups by two tailed unpaired t-test. Scale bar indicates 50 μm. (FIG. 6 panel H) The number of lung tissues with or without TIL was compared between the two groups by the Chi-square test. (FIG. 6 panel I) The weight of mice treated with PBS or IL2-ep13nsEV4T1 was measured and compared by two tailed unpaired t test at different time points. (FIG. 6 panel J) The ALT and AST in the serum of mice were measured and compared by unpaired t test. (FIG. 6 panel K) The cytokines in the blood of mice in control group and IL2-ep13nsEV4T1 treatment group were measured by ELISA and compared by unpaired t tests. n.s. P≥0.05, *P<0.05, **P<0.01.

FIG. 7 shows an image of a schematic illustrating an embodiment of the design of IL2-ep13nsEV and its utilization in treating breast cancer. To generate this novel active immunotherapy, the sEVs from autologous DCs are engineered with surface membrane-bound IL2 by expressing IL2-MFG-E8. This personalization of DC-derived sEV (p13nsEV) is achieved by loading lysed surgically harvested breast cancer cells onto engineered autologous DCs followed by collecting sEVs that are then used as personalized immunotherapy. Importantly, we found that LPS and STING agonist worked together to promote the expression of co-stimulatory factors on the surface of this engineered vesicle. Therefore, this sEV geared with tumor lysate-derived antigens, bioactive membrane-bound IL2, and enhanced with co-stimulatory factors, is named as “IL2-ep13nsEV”. IL2-ep13nsEV is designed to act as active immunotherapy to expand the pool of cancer-specific immune cells by facilitating neoantigen processing and presentation, as well as T cell activation. It can be used to prevent the recurrence of surgically removed primary tumor, or to treat advanced breast cancer resistant to ICI.

FIG. 8 panels A-D show images of data plots and schematics relating to subtractive hybridization to enrich tumor-specific mRNA for personalized DCSEV. (FIG. 8 panel A) OVA mRNA was transfected to DC. H-2Hb/SIINFEKL on DC and DCSEV were measured and compared by flow cytometry. (FIG. 8 panel B) The principle of subtractive hybridization to enrich tumor-specific mRNA. (FIG. 8 panel C) The expression of representative genes was measured for the RNA prepared from EO771, breast epithelial cells from C57BL/6J mice, and the subtracted RNA by real-time PCR using spike-in control. (FIG. 8 panel D) IL2mDCSEV isolated from DC that were pulse-educated with PBS, scrambled RNAs or the subtracted RNAs was used to prime the T cells, that were later used for cytotoxicity assay on EO771 cells. The cytotoxicity was measured by Aqua Zombie⁺ dead cells among GFP⁺ cancer cells.

FIG. 9 panels A-D show images of data plots and schematics related to subtractive hybridization to enrich tumor-specific mRNA for personalized DCSEV. (FIG. 9 panel A) OVA mRNA constructed IL2mDCsEV were used to prime to T cells, which were used for cytotoxicity assay with B16-OVA cells. Dead B16-OVA cells were quantified by flow cytometry. (FIG. 9 panel B) OVA mRNA constructed IL2mDCsEV were used to treat B16-OVA tumors in mice. The growth of primary tumors in mice was tracked. (FIG. 9 panel C) TCR sequencing was performed for TIL in the tumors. (FIG. 9 panel D) Clonotypes of the T cells were quantified by TCR sequencing.

FIG. 10 panels A-E show images of tissue and data plots related to combining the subtractive hybridization and DCsEV bio-engineering to generate IL2/co-sEV^{mRNA}. (FIG. 10 panel A) The subtracted or non-subtracted mRNAs were loaded onto mBMDC that express IL2-MFG-E8, followed by inducing DC maturation and treatment with STING agonist, Cyclic diGMP (c-diGMP). The IL2/co-sEV^{mRNA} was isolated from the DC by differential centrifugation, and they were used for the treatment of C57BL/6J mice with E0771 tumor, at five different time points. Tumors at the endpoint were shown. (FIG. 10 panel B) Tumor growth on mammary fat pad was monitored by measuring the tumor size by a caliper. The unpaired t-test was performed to compare the tumor sizes at different time points. (FIG. 10 panel C) The tumor weight at the end point (Day 25) is shown for the two different treatment groups. Unpaired t test was performed to compare the tumor weight. (FIG. 10 panel D) The IFN-γ+/CD3+, CD4+ and CD8+ cells in dissociated splenocytes were examined by flow cytometry. The percentage of cells was compared between the two treatment groups by the unpaired t test. (FIG. 10 panel E) The tumors were dissociated, and CD4+ and CD8+ TIL were measured by flow cytometry for each group, and unpaired t-test was performed to compare the percentage of TIL.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “recombinant nucleic acid,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

As used herein, the phrase “substantially identical,” or “substantial identity” in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, or more nucleotides in length, and any range therein, up to the full length of the sequence. In some embodiments, the nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40 nucleotides). In some embodiments, a substantially identical nucleotide or protein sequence performs substantially the same function as the nucleotide (or encoded protein sequence) to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Two nucleotide sequences may also be considered substantially complementary when the two sequences hybridize to each other under stringent conditions. In some representative embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

As used herein, an “isolated” polynucleotide (e.g., an “isolated DNA” or an “isolated RNA”) means a polynucleotide at least partially separated from at least some of the other components of the naturally occurring organism or virus, for example, the cell or viral structural components or other polypeptides or nucleic acids commonly found associated with the polynucleotide. In representative embodiments an “isolated” nucleotide is enriched by at least about 10-fold, 100-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

A “nucleic acid,” “nucleic acid molecule,” or “nucleotide sequence” is a sequence of nucleotide bases, and may be RNA, DNA or DNA-RNA hybrid sequences (including both naturally occurring and non-naturally occurring nucleotide), but is preferably either single or double stranded DNA sequences.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

As used herein, the term “chimera,” “chimeric,” and/or “fusion protein” refer to an amino acid sequence (e.g., polypeptide) generated non-naturally by deliberate human design comprising, among other components, an amino acid sequence of a protein of interest and/or a modified variant and/or active fragment thereof (a “backbone”), wherein the protein of interest comprises modifications (e.g., substitutions such as singular residues and/or contiguous regions of amino acid residues) from different wild type reference sequences (chimera), optionally linked to other amino acid segments (fusion protein). For example, a fusion protein is a polypeptide produced when two (or more) heterologous nucleotide sequences or fragments thereof coding for two (or more) different polypeptides not found fused together in nature are fused together in the correct translational reading frame. The different components of the designed protein may provide differing and/or combinatorial function. Structural and functional components of the designed protein may be incorporated from differing and/or a plurality of source material. The designed protein may be delivered exogenously to a subject, wherein it would be exogenous in comparison to a corresponding endogenous protein.

The term “endogenous” refers to a component naturally found in an environment, i.e., a gene, nucleic acid, miRNA, protein, cell, or other natural component expressed in the subject, as distinguished from an introduced component, i.e., an “exogenous” component.

A “therapeutic,” “therapeutic polypeptide,” “therapeutic molecule” and similar terms refer to a polypeptide and/or molecule that can alleviate, reduce, prevent, delay and/or stabilize symptoms that result from an absence or defect in a protein in a cell or subject and/or is a polypeptide and/or molecule that otherwise confers a benefit to a subject, e.g., anti-cancer effects or improvement in transplant survivability or induction of an immune response.

A “recombinant” nucleic acid, polynucleotide or nucleotide sequence is one produced by genetic engineering techniques.

A “recombinant” polypeptide is produced from a recombinant nucleic acid, polypeptide or nucleotide sequence.

As used herein with respect to nucleic acids, the term “fragment” refers to a nucleic acid that is reduced in length relative to a reference nucleic acid and that comprises, consists essentially of and/or consists of a nucleotide sequence of contiguous nucleotides identical or almost identical (e.g., 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. In some embodiments, the nucleic acid fragment comprises, consists essentially of or consists of at least about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive nucleotides. In some embodiments, the nucleic acid fragment comprises, consists essentially of or consists of less than about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive nucleotides.

As used herein with respect to polypeptides, the term “fragment” refers to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive amino acids. In some embodiments, the polypeptide fragment comprises, consists essentially of or consists of less than about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive amino acids.

As used herein with respect to nucleic acids, the term “functional fragment” or “active fragment” refers to nucleic acid that encodes a functional fragment of a polypeptide.

As used herein with respect to polypeptides, the term “functional fragment” or “active fragment” refers to polypeptide fragment that retains at least about 20%, 25%, 30%, 35%, 40%, 45%, 50%, 55%, 60%, 65%, 70%, 75%, 80%, 85%, 90%, 95%, 96%, 97%, 98%, 99%, 99.5% or more of at least one biological activity of the full-length polypeptide (e.g., the ability to up- or down-regulate gene expression). In some embodiments, the functional fragment actually has a higher level of at least one biological activity of the full-length polypeptide.

As used herein, the term “modified,” as applied to a polynucleotide or polypeptide sequence, refers to a sequence that differs from a wild-type sequence due to one or more deletions, additions, substitutions, or any combination thereof. Modified sequences may also be referred to as “modified variant(s).”

The terms “immunogen” and “antigen” are used interchangeably herein and mean any compound (including polypeptides) to which a cellular and/or humoral immune response can be directed. In particular embodiments, an immunogen or antigen can induce a protective immune response against the effects of cancer.

“Pharmaceutically acceptable” as used herein means that the compound, carrier, or composition is suitable for administration to a subject to achieve a treatment described herein, without unduly deleterious side effects in light of the severity of the disease and necessity of the treatment.

As used herein, the terms “subject” and “patient” are used interchangeably herein and refer to both human and nonhuman animals. A subject of this invention can be any subject that is susceptible to a disorder that can benefit by the methods and compositions of the present invention and/or be treated for a disorder by the methods and compositions of the present invention. In some embodiments, the subject of any of the methods of the present invention is a mammal. The term “mammal” as used herein includes, but is not limited to, humans, primates, non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. As a further option, the subject can be a laboratory animal and/or an animal model of disease. Preferably, the subject is a human. The subject may be of any gender, any ethnicity and any age.

A method of the present invention may also be carried out on animal subjects, particularly mammalian subjects such as mice, rats, dogs, cats, livestock and horses for veterinary purposes, and/or for drug screening and drug development purposes.

In some embodiments, the subject is “in need of” or “in need thereof” a method of the present invention, for example, the subject has findings typically associated with cancer (e.g., breast cancer, lung cancer, and the like).

As used herein, the term “therapeutically effective amount” refers to an amount of an isolated vesicle and/or a composition of the present invention that elicits a therapeutically useful response in a subject. Those skilled in the art will appreciate that the therapeutic effects need not be complete or curative, as long as some benefit is provided to the subject.

“Treat,” “treating” or “treatment of” (and grammatical variations thereof) as used herein refer to any type of treatment that imparts a benefit to a subject and may mean that the severity of the subject's condition is reduced, at least partially improved or ameliorated and/or that some alleviation, mitigation or decrease in at least one clinical symptom associated with the subject's condition (e.g., cancer) is achieved and/or there is a delay in the progression of the symptom. In some embodiments, the severity of a symptom associated with the subject's condition (e.g., cancer) may be reduced in a subject compared to the severity of the symptom in the absence of a method of the present invention.

A “treatment effective” amount as used herein is an amount that is sufficient to treat (as defined herein) a subject. Those skilled in the art will appreciate that the therapeutic effects need not be complete or curative, as long as some benefit is provided to the subject. In some embodiments, a treatment effective amount may be achieved by administering a composition of the present invention.

The terms “prevent,” “preventing” and “prevention” (and grammatical variations thereof) refer to avoidance, reduction and/or delay of the onset of a symptom associated with the subject's condition (e.g., cancer) relative to what would occur in the absence of a method of the present invention. The prevention can be complete, e.g., the total absence of the symptom. The prevention can also be partial, such that the occurrence of the symptom in the subject and/or the severity of onset is less than what would occur in the absence of a method of the present invention.

A “prevention effective” amount as used herein is an amount that is sufficient to prevent (as defined herein) a symptom associated with the subject's condition (e.g., cancer). Those skilled in the art will appreciate that the level of prevention need not be complete, as long as some benefit is provided to the subject. In some embodiments, a prevention effective amount may be achieved by administering a composition of the present invention.

The term “contacting”, “incubating” and/or “culturing” as used herein indicates actions directed to creation of a spatial relationship between two items provided for a time and under conditions such that at least one of the reciprocal or non-reciprocal action or influence between the two items can be exerted. In particular, incubation can be performed between a substance and a cell and can result in a direct contact and/or interaction between the substance and the cell or can result in a modification of the cell following an indirect action of the bacterial substance (e.g. following activation or modification of another substance which directly interacts with the cell).

The term “administering” or “administered” as used herein is meant to include topical, parenteral and/or oral administration, all of which are described herein. Parenteral administration includes, without limitation, intravenous, subcutaneous and/or intramuscular administration (e.g., skeletal muscle or cardiac muscle administration). It will be appreciated that the actual method and order of administration will vary according to, inter alia, the particular preparation of compound(s) being utilized, and the particular formulation(s) of the one or more other compounds being utilized. The optimal method and order of administration of the compositions of the invention for a given set of conditions can be ascertained by those skilled in the art using conventional techniques and in view of the information set out herein. The term “administering” or “administered” also refers, without limitation, to oral, sublingual, buccal, transnasal, transdermal, rectal, intramuscular, intravenous, intraarterial (intracoronary), intraventricular, intrathecal, and subcutaneous routes. In accordance with good clinical practice, the instant compounds can be administered at a dose that will produce effective beneficial effects without causing undue harmful or untoward side effects, i.e., the benefits associated with administration outweigh the detrimental effects.

The terms “protective” immune response or “protective” immunity as used herein indicates that the immune response confers some benefit to the subject in that it prevents or reduces the incidence and/or severity and/or duration of disease or any other manifestation of an infection or nonself stress. For example, in representative embodiments, a protective immune response or protective immunity results in enhanced natural killer (NK) cells and/or NK cell anti-tumor responses (e.g., expanded population of NK cells, enhanced population of infiltrating NK cells, enhanced expression of granzyme B (gzmB+), and/or expanded population of gzmB+ NK cells in the tumor microenvironment), whether or not accompanied by clinical disease. Alternatively, a protective immune response or protective immunity may be useful in the therapeutic treatment of existing disease.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to antigens the host views as “non-self”, e.g., immunogens by infection or by vaccination, e.g., cancer antigens. Active immunity can be contrasted with passive immunity, which is acquired through the “transfer of preformed substances (antibody, transfer factor, thymic graft, interleukin-2) from an actively immunized host to a non-immune host.” Id.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

As used herein, “immunosuppressive” refers to a function or activity that suppresses one or more aspects of an active immune response. An “immunosuppressive agent” refers to an agent that inhibits or prevents an immune response, e.g., to a foreign material in a subject. Immunosuppressive agents generally act by inhibiting immune cell (e.g., T-cell, B-cell, NK cell, and the like) activation, disrupting proliferation, and/or suppressing inflammation.

The term “biologically active” as used herein means an enzyme or protein having structural, regulatory, or biochemical functions of a naturally occurring molecule.

The terms “antibody” and “immunoglobulin” include antibodies or immunoglobulins of any isotype, fragments of antibodies that retain specific binding to antigen, including but not limited to Fab, Fv, single chain Fv (scFv), Fc, and Fd fragments, chimeric antibodies, humanized antibodies, single-chain antibodies, and fusion proteins including an antigen-binding portion of an antibody and a non-antibody protein. The antibodies can in some embodiments be detectably labeled, e.g., with a radioisotope, an enzyme which generates a detectable product, a fluorescent protein, and the like. The antibodies can in some embodiments be further conjugated to other moieties, such as members of specific binding pairs, e.g., biotin (member of biotin-avidin specific binding pair), and the like. Also encompassed by the terms are Fab′, Fv, F(ab′)₂, and other antibody fragments that retain specific binding to antigen (e.g., any antibody fragment that comprises at least one paratope).

Antibodies can exist in a variety of other forms including, for example, Fv, Fab, and (Fab′)₂, as well as bi-functional (i.e., bi-specific) hybrid antibodies (see e.g., Lanzavecchia et al., 1987) and in single chains (see e.g., Huston et al., 1988 and Bird et al., 1988, each of which is incorporated herein by reference in its entirety). See generally, Hood et al., 1984, and Hunkapiller & Hood, 1986. The phrase “detection molecule” is used herein in its broadest sense to include any molecule that can bind with sufficient specificity to a biomarker to allow for detection of the particular biomarker. To allow for detection can mean to determine the presence or absence of the particular biomarker member and, in some embodiments, can mean to determine the amount of the particular biomarker. Detection molecules can include antibodies, antibody fragments, and nucleic acid sequences.

As used herein, the term “sample” is used in its broadest sense. In one sense, it is meant to include a specimen from a biological source. A “sample” or “biological sample” of this invention can be any biological material, such as a biological fluid, an extract from a cell, an extracellular matrix isolated from a cell, a cell (in solution or bound to a solid support), a tissue, a tissue homogenate, and the like as are well known in the art. For example, biological samples can be obtained from animals (including humans) and encompass fluids (e.g., blood, mucus, urine, saliva), solids, tissues, cells, and gases. In some embodiments, the sample is obtained from a tumor (e.g., tumor stroma) in the subject. The sample may also comprise one or more immune cells, including T cells of the subject, including immune cells (e.g., helper T cells) from the tumor (e.g., tumor stroma) of the subject.

As used herein the term “control” refers to a comparative sample and/or other reference source for a control subject.

“Control subject” as used herein refers to a subject which does not have said condition(s) of the subject in need, e.g., said cancer and/or an illness to which the methods of the present invention disclosed herein may provide beneficial health effects.

As used herein, “Stimulator of Interferon Genes” or “STING” refers to a transmembrane protein which resides in the endoplasmic reticulum and is also known as TMEM173, MITA, ERIS and/or MPYS. STING is a component of the cGAS-STING cytosolic DNA sensing pathway of the innate immune system. While not wishing to be bound to theory, STING activation is believed to occur upon entry of double-stranded DNA into the cytosol of a cell (e.g., a host cell). STING activators (e.g., agonists) include native and synthetic agonists such as, but not limited to, cyclic CMP-AMP (“cGAMP”), G10, diABZI, ADU-S100, and/or MSA-2. Further description of STING and its activities can be found in Corrales and Gajewski, 2015 Clin Cancer Res 21(21):4774-4779; Hopfner and Hornung 2020 Nat Rev Mol Cell Biol 21:501-521; and Decout et al. 2021 Nat Rev Immunol 21:548-569, the disclosures of each of which are incorporate herein by reference.

A “vector” refers to a compound used as a vehicle to carry foreign genetic material into another cell, where it can be replicated and/or expressed. A cloning vector containing foreign nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene. Insertion of a vector into the target cell is referred to transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

The term “vesicle”, “extracellular vesicle” and “EV” may be used interchangeably herein to refer to lipid bound vesicles secreted by cells into the extracellular space. While not wishing to be bound to theory, EVs are proposed to originate from the inward budding of endosomes into the multivesicular body (MVB) to create intraluminal vesicles (ILVs), which then traffic to the plasma membrane to release the exosomes as a cluster (Pegtel and Gould, 2019 Annu Rev Biochem 88:487-514, incorporated herein by reference). Ab EV can include, but are not limited to, an exosome, an ectosome, a microvesicle, nanovesicle and/or an exosome-like vesicle. The three main subtypes of EVs are microvesicles (MVs), exosomes and apoptotic bodies. Exosomes are the smallest EV subgroup, ranging in size from 40-150 nanometers (nm). Their surfaces are marked with tetraspanin proteins such as CD9, CD63, and CD81, and internalized proteins comprise many members of the endosomal sorting complex required for transport (ESCRT) machinery (Cocozza et al. 2020 Cell 182(1):262; Thery et al. 2018 J Extracell Vesicles 7(1):1535750). EVs may carry “cargo” within the vesicle membrane, including but not limited to, lipids, nucleic acids and proteins. Proteins associated with EVs are typically associated with the plasma membrane (e.g., the surface membrane) of the secreting cell, as well as those of the cytosol or those involved in lipid metabolism. EVs, their subtypes and biological uses are further described and reviewed in Doyle and Wang 2019, Cells 8:727, the disclosures of which are incorporated herein by reference in its entirety.

The term “antigen presenting cell” as used herein refers to a cell that displays foreign antigen complex with major histocompatibility complex (MHC; e.g., MHC-I and/or MHC-II; referred to in humans as “human leukocyte antigen” or “HLA” molecules) on its surface. In some embodiments, an antigen presenting cell may comprise a dendritic cell, a macrophage, a B cell and/or additional cells identifiable by a skilled person.

Anticancer immunotherapies can be categorized into either “passive” or “active” immunotherapy, even though the principle of the categorization is inconsistent among the different sources of the reports (Galluzzi et al. 2014 Oncotarget 5:12472-12508). From the standpoint of the definition based on their reliance on the intrinsic anti-tumor immunity, immune checkpoint inhibitors (“ICIs”) are considered passive immunotherapies with their need for pre-existing anti-tumor T cells. ICIs, designed to augment the anti-tumor function of T cells, do not effectively function in patients with difficulty in generating reactive immune cells. To incite a successful immune response to cancer cells, various active immunotherapies have been developed to increase the presence of tumor-targeted immune cells. In the past decades, pre-clinical studies and early phase clinical trials have been carried out to test such active immunotherapies, including cancer vaccines and adoptive cell therapies (ACT), for treating malignant diseases. While these studies have proven the potential capability of these therapies in promoting the cross-presentation and generating active T cells, the results of phase III clinical have been disappointing. The failure of clinical impact can be attributable to several flaws of these active immunotherapies. Some cancer vaccines take advantage of increased gene mutations in cancer cells by targeting selected mutated genes to treat cancer. However, due to high tumor heterogeneity, resistant clones are quickly enriched among cancer cells that do not express the targeted genes (Alcazer et al. 2019 Eur J of Cancer 108:55-60).

Another important cause of failure is the lack of inclusion of ICIs in past clinical trials. Despite the vaccine-induced immune response, cancer cells are still able to evade immune cells by expressing immune checkpoints. Other active immunotherapies based on the adoptive transfer of live cells, including both dendritic cells (Boudewijns et al. 2020 Cancer Immunol Immunother 69:477-488) and T cells (Bernhard et al. 2008 Cancer Immunol Immunother 57:271-280), have also faced challenges when tested in treating patients with solid tumors. These cells are vulnerable to the stress of in vitro cell culture (Maurer et al. 2020 Cancer Immunol Res 8:1554-1567) as well as cancer cell-induced immunosuppressive microenvironment in vivo (Saxena and Bhardwaj 2018 Trends Cancer 4:119-137). It was reported that adoptively transferred DCs had dysregulated co-stimulatory factors, resulting in compromised T cell priming and unbeneficial treatment effect (Bol et al. 2020 Annals of Oncology 31:S732). For the approach of using tumor-infiltrating lymphocytes (TIL), it was found that loss of tumor-reactive T-cell clones occurred during the in vitro expansion (Poschke et al. 2002 Clinical Cancer Research 26:4289-4301). Moreover, the intratumoral T cells have lost the repertoire before extraction for expansion due to their exhausted phenotype (Scheper et al. 2019 Nature Medicine 25:89-94).

The use of live cells also adds impediment to the manufacturing, storage, and transportation of such therapeutics, which will greatly impact the viability of the product. Difficulty in cryopreservation and long-term storage can also lead to batch inconsistency, decreased cell recovery and functionality (Morotti et al. 202 Br J Cancer 124:1759-1776). The low viability of the adoptively transferred cells eventually causes an unsatisfactory immune response. Serious adverse events are another obstacle that prevents the translational application of ACT in treating cancer. For example, engineered CAR-T cells were found to cause life-threatening symptoms, including multiple cardiac arrests, respiratory distress, as well as multi-organ damage due to uncontrolled inflammatory cytokine release (Morgan et al. 2010 Molecular Ther 18:843-851).

Breast cancer has the highest incidence rate in the U.S. compared to other types of cancers. While non-metastatic breast cancer can be treated with surgery and chemotherapies, around 22% of breast cancer patients eventually experience recurrence within 10 years. For the breast cancer patients at an advanced stage, while systemic endocrine and/or chemotherapies are available, these therapies fall behind in saving the patients, with a 10-year survival rate at around 13. Thus, better therapies are needed for treating patients at advanced stages and for the prevention of recurrent disease. Recently, immune therapies such as ICIs have emerged as alternatives for treating multiple types of solid tumors. However, compared to other types of cancers, breast cancer is considered immunogenically ‘cold’. Unlike other ‘hot tumors’ such as lung cancer and melanoma which usually contain more than two hundred non-synonymous mutations (Castle et al. 2019 Frontiers in Immunol 10:1856), breast cancer cells have a lower tumor mutation burden with barely more than thirty non-synonymous mutations. Breast cancer cells also impede the function of APCs. It was reported that breast cancer cells can suppress the maturation and function of DCs by direct cell-cell interaction through CTLA-4 expressed cancer cells (Chen et al. 2017 Oncotarget 8:13703-13715). Furthermore, breast cancer cells often negatively regulate APC through secreted factors. Putrescine, a polyamine molecule released by cancer cells, was found to suppress the maturation of DCs and the expression of MHC II on DCs. Breast cancer cells were also found to promote the expansion of TAMs (tumor-associated macrophages) and mobilize the release of IL-10 from TAMs to repress the DCs (Ruffell et a. 2014 Cancer cell 26:623-637). Moreover, extracellular vesicles have been found to assist the tumor progression by modulating the immune microenvironment, and sEVs of breast cancer were found to educate and skew DCs into a pro-tumor phase through HSP72 and HSP105 (Shen et al. 2017 Oncoimmunology 6:e1362527). With these limitations, APCs lose the ability to present the neoantigens and prime the T cells. Accordingly, the use of ICIs in breast cancer faces challenges due to a lack of reactive T lymphocytes.

The inventors of the present invention aimed to develop an active immunotherapy using nanovesicles derived from dendritic cells that were engineered to (i) express bioactive IL2, (ii) strongly express co-stimulatory factors, and (iii) personalizeable to treat individual tumors (FIG. 7). Without wishing to be bound to theory, the expression of bioactive IL2 on the surface led to a significant increase in the interaction of the engineered vesicle with the T cells. It was also found that LPS and STING agonist work together to induce the expression of co-stimulatory factors on the surface of the nanovesicle which strongly augmented T-cell activation. These characteristics provide an active immunotherapy that is able to perform autonomous T cell priming independent of other APC, and suppress the growth of cancer cells such as but not limited to breast cancer. As further described in the examples, this active immunotherapy significantly inhibited the growth of a breast cancer cell (E0771) that has a high mutation burden but also the growth of an “immune-cold” breast tumor cell (4T1) which has low mutation burden and weak T cell infiltration (Yang et al. 2017 Oncotarget 8:30621-30643). Furthermore, this therapeutic also reversed the ICI resistance of 4T1 cells and presented a suppressive effect on the recurrence of tumor in distant organs.

ICIs have been shown to be effective only for limited types of cancers and a small fraction of patients. Although the response to ICIs is relatively quick if working, it is not long-lasting. Patients often develop resistance to ICIs after the initial response. To generate more effective and long-lasting treatment, active immunotherapies are needed to induce immune cell memory. Currently, only a handful of active immunotherapies have been approved to treat solid tumors. Preventive cancer vaccines were approved to treat cervical and liver cancers. However, these active immunotherapies are targeting oncogenic viruses instead of cancer cells. Another approved active immunotherapy is Sipuleucel-T (Cheever and Higano 2011 Clinical cancer research 17:3520-3526), which is an adoptive DC therapy for the treatment of metastatic castration-resistant prostate cancer. This therapy is based on the prostatic acid phosphatase protein that is highly expressed in prostate cancer cells. Such an approach that is focused on a single tumor antigen has a challenge for the treatment of cancers with inherent heterogeneity, and clonal selection may lead to the emergence of resistant tumor cells. To overcome this issue, the inventors of the present invention utilized total tumor lysate as the source to generate the extracellular vesicles (FIG. 7). Thus, without wishing to be bound to theory, the products and processes described here may work as personalizeable treatments for breast cancer patients by targeting the whole population of malignant cells. Furthermore, the tumor lysates include both tumor-specific proteins and mRNAs that serve as the source to generate the repository of lymphocytes. It was reported that tumor mRNA loaded into nanoparticles was able to give rise to T cells that were reactive to the mRNA-specific antigens (Sahin et al. 2020 Nature 585:107-112). Such an approach indeed was utilized for COVID-19 and has been proven to be effective.

In the studies described herein, instead of using lipid nanoparticles, the inventors engineered EVs from DCs so that these vesicles could present both membrane-bound IL2 and T cell priming machinery including but not limited to MHC I, MHC II, 4-1BBL, and CD40L. IL2 is also called T cell growth factor, and it plays a central role in regulating the T cell development, expansion, and homeostasis. It is a key cytokine that is important for the growth of both effector and regulatory T cells (Kalia and Sarkar 2018 Frontiers in immunology 9:2987).

Accordingly, one aspect of the present invention provides an isolated nonnucleated extracellular vesicle (EV) comprising a surface membrane and a fusion protein comprising an interleukin-2 (IL-2) molecule and/or functional fragment thereof, a major histocompatibility (MHC) molecule and/or functional fragment thereof (e.g., MHC-I; e.g., MHC-II), and one or more costimulatory molecule, each expressed on the surface membrane of the EV; wherein the MHC molecule and/or functional fragment thereof presents an exogenously introduced antigen.

The term “presents” as used herein refers to the appropriate binding of the antigen to the MHC-I and/or MHC-II molecule such that it can be adequately presented to a cognate T cell and/or B cell receptor (TCR and/or BCR) on an immune cell, e.g., an immune cell in and/or of the subject.

The exogenously introduced antigen may be any antigen or mixed population of two or more antigens of interest in stimulating an immune response against in the subject. In some embodiments, the exogenously introduced antigen is a cancer antigen (e.g., a tumor antigen, e.g., a cancer neoantigen).

The EV of the present invention may be any type and/or sized extracellular vesicle secreted or secretable by an antigen present cell. In some embodiments, the EV of the present invention may be, but is not limited to, an exosome, an ectosome, a microvesicle, nanovesicle and/or an exosome-like vesicle.

In some embodiments, the EV may be about 50 nm in diameter to about 500 nm in diameter, e.g., about 10, 50, 100, 150, 200, 250, 300, 350, 400, 450, or 500 nm in diameter or any value or range therein. For example, in some embodiments, the isolated EV may be about 50 to about 500 nm in diameter, about 100 to about 200 nm in diameter, about 10 to about 250 nm in diameter, about 75 to about 125 nm in diameter, or about 50 nm, about 100 nm, about 110 nm, about 115 nm, about 120 nm, about 150 nm, about 200 nm, about 250 nm, or about 500 nm in diameter.

The one or more costimulatory molecule may be any costimulatory molecule known in the art or later discovered. In some embodiments, the one or more costimulatory molecule is a molecule induced by stimulation of the cGAS-STING and/or the TLR innate immune signaling pathways. In some embodiments, the one or more costimulatory molecule may be 4-1BB, CD40L and/or Tim3.

In some embodiments, the EV may be isolated from an antigen presenting cell (APC) of a mammalian subject. In some embodiments, the EV may be isolated from an APC from a healthy subject. In some embodiments, the EV may be isolated from an APC from a patient (e.g., a subject in need thereof).

In some embodiments, the exogenously introduced antigen may be isolated from a mammalian subject (e.g., a subject in need thereof, e.g., a patient).

In some embodiments, the EV and the introduced antigen are isolated from the same mammalian subject (e.g., subject in need; e.g., patient).

Also provided herein is a composition comprising the isolated EV of the present invention. In some embodiments, a composition of the present invention may further comprise a pharmaceutically acceptable carrier, diluent, and/or adjuvant.

By “pharmaceutically acceptable” it is meant a material that is not toxic or otherwise undesirable, i.e., the material may be administered to a subject without causing any undesirable biological effects. For injection, the carrier will typically be a liquid. For other methods of administration (e.g., such as, but not limited to, administration to the mucous membranes of a subject (e.g., via intranasal administration, buccal administration and/or inhalation)), the carrier may be either solid or liquid. For inhalation administration, the carrier will be respirable, and will preferably be in solid or liquid particulate form. The formulations may be conveniently prepared in unit dosage form and may be prepared by any of the methods well known in the art. In some embodiments, that pharmaceutically acceptable carrier can be a sterile solution or composition.

In some embodiments, the present invention provides a pharmaceutical composition comprising the isolated EV of the present invention, a pharmaceutically acceptable carrier, and, optionally, other medicinal agents, therapeutic agents, pharmaceutical agents, stabilizing agents, buffers, carriers, adjuvants, diluents, etc., which can be included in the composition singly or in any combination and/or ratio.

Immunogenic compositions comprising the isolated EV of the present invention may be formulated by any means known in the art. Such compositions, especially vaccines, are typically prepared as injectables, either as liquid solutions or suspensions. Solid forms suitable for solution in, or suspension in, liquid prior to injection may also be prepared. Lyophilized preparations are also suitable. In some embodiments, a pharmaceutical composition of the present invention may be a vaccine formulation, e.g., may comprise an isolated EV of the present invention and adjuvant(s), optionally in a vaccine diluent. The active immunogenic ingredients are often mixed with excipients and/or carriers that are pharmaceutically acceptable and/or compatible with the active ingredient. Suitable excipients include but are not limited to sterile water, saline, dextrose, glycerol, ethanol, or the like and combinations thereof, as well as stabilizers, e.g., HSA or other suitable proteins and reducing sugars. In addition, if desired, the vaccines or immunogenic compositions may contain minor amounts of auxiliary substances such as wetting and/or emulsifying agents, pH buffering agents, and/or adjuvants that enhance the effectiveness of the vaccine or immunogenic composition.

In some embodiments, a pharmaceutical composition comprising the isolated EV of the present invention may further comprise additional agents, such as, but not limited to, additional antigen as part of a cocktail in a vaccine, e.g., a multi-component vaccine wherein the vaccine may additionally include peptides, cells, virus, viral peptides, inactivated virus, etc.

In some embodiments, a pharmaceutical composition comprising the isolated EV of the present invention, and a pharmaceutically acceptable carrier may further comprise an adjuvant. As used herein, “suitable adjuvant” describes an adjuvant capable of being combined with a an isolated EV of the present invention to further enhance an immune response without deleterious effect on the subject or the cell of the subject.

The adjuvants of the present invention can be in the form of an amino acid sequence, and/or in the form or a nucleic acid encoding an adjuvant. When in the form of a nucleic acid, the adjuvant can be a component of a nucleic acid encoding the polypeptide(s) or fragment(s) or epitope(s) and/or a separate component of the composition comprising the nucleic acid encoding the polypeptide(s) or fragment(s) or epitope(s) of the invention. According to the present invention, the adjuvant can also be an amino acid sequence that is a peptide, a protein fragment or a whole protein that functions as an adjuvant, and/or the adjuvant can be a nucleic acid encoding a peptide, protein fragment or whole protein that functions as an adjuvant. As used herein, “adjuvant” describes a substance, which can be any immunomodulating substance capable of being combined with a composition of the invention to enhance, improve, or otherwise modulate an immune response in a subject.

In further embodiments, the adjuvant can be, but is not limited to, an immunostimulatory cytokine (including, but not limited to, GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules), SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but may also be a salt of calcium, iron or zinc, or may be an insoluble suspension of acylated tyrosine, or acylated sugars, cationically or anionically derivatized polysaccharides, or polyphosphazenes.

Other adjuvants are well known in the art and include without limitation MF 59, LT-K63, LT-R72 (Pal et al. Vaccine 24(6):766-75 (2005)), QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153, or a less reactogenic composition where the QS21 is quenched with cholesterol as disclosed in PCT publication number WO 96/33739. A particularly potent adjuvant formulation involving QS21 3D-MPL & tocopherol in an oil in water emulsion is described in PCT publication number WO 95/17210. In addition, the nucleic acid compositions of the invention can include an adjuvant by comprising a nucleotide sequence encoding the antigen and a nucleotide sequence that provides an adjuvant function, such as CpG sequences. Such CpG sequences, or motifs, are well known in the art.

Adjuvants can be combined, either with the compositions of this invention or with other vaccine compositions that can be used in combination with the compositions of this invention.

Also provided herein is an isolated cell (e.g., an immortalized cell, e.g., a cell line) comprising the EV and/or composition of the present invention. In some embodiments, the isolated cell may comprise an APC (e.g., an APC cell line) generated according to the methods as described herein to secrete an EV of the present invention.

Also provided herein is a kit comprising a composition of the present invention, and optional instructions for the use thereof.

Kits that include particles of this invention and/or a pharmaceutical composition as described herein are also provided herein. Some kits include particles and/or compositions in a container (e.g., vial or ampule), and may also include instructions for use of the particles and/or composition in the various methods disclosed above. The particles and/or composition can be in various forms, including, for instance, as part of a solution or as a solid (e.g., lyophilized powder). The instructions may include a description of how to prepare (e.g., dissolve or resuspend) the particles in an appropriate fluid and/or how to administer the particles for the treatment of the diseases and disorders described herein.

The kits may also include various other components, such as buffers, salts, complexing metal ions and other agents described above in the section on pharmaceutical compositions. These components may be included with the chimeric protein or may be in separate containers. The kits may also include other therapeutic agents for administration with the chimeric protein. Examples of such agents include, but are not limited to, agents to treat the disorders or conditions described above.

Another aspect of the invention provides a method of producing an immune response to a disorder in a subject, comprising administering to the subject an effective amount of the isolated EV and/or the composition of the present invention.

Another aspect of the invention provides a method of protecting a subject from the effects of a disorder, comprising administering to the subject an effective amount of the isolated EV and/or the composition of the present invention.

In some embodiments, the subject is at risk for or suspected to have or develop the disorder.

In some embodiments, administering to the subject the effective amount of the isolated EV and/or the composition to the subject may occur prior to the subject developing symptoms of the disorder (e.g., administering prophylactically, e.g., as a prophylactic vaccine).

Another aspect of the invention provides a method of treating a disorder in a subject, comprising administering to the subject (e.g., the subject having or suspected of having or developing the disorder) an effective amount of the isolated EV and/or the composition of the present invention.

The disorder may be any disorder to which the methods and compositions described herein may be of therapeutic benefit. In some embodiments, the disorder may be cancer. In some embodiments, the cancer may be breast cancer, including but not limited to, BRCA⁺ breast cancer, HER⁺ breast cancer, progesterone receptor (PR)⁺ breast cancer, triple-negative (BRCA⁻/HER⁻/PR⁻) breast cancer, lobular breast carcinoma, ductal breast carcinoma, breast adenocarcinoma, and/or metastatic breast cancer. In some embodiments, the cancer may be recurrence of cancer in another site other than the breast (e.g., the lungs, the head and neck, the liver, the kidneys, the lymph nodes, etc.), wherein the cancer originates from metastatic breast cancer.

Another aspect of the invention provides a method of producing an immune response to a cancer in a subject, comprising administering to the subject an effective amount of the isolated EV and/or the composition of the present invention.

Another aspect of the invention provides a method of preventing a disorder associated with a cancer in a subject, comprising administering to the subject an effective amount of the isolated EV and/or the composition of the present invention.

Another aspect of the invention provides a method of protecting a subject from the effects of a cancer, comprising administering to the subject an effective amount of the isolated EV and/or the composition of the present invention.

In some embodiments, the subject may be at risk for or suspected to have or develop a cancer.

In some embodiments, administering the effective amount of the isolated EV and/or composition to the subject may occur prior to the subject developing symptoms of the cancer (e.g., administering prophylactically, e.g., as a prophylactic vaccine).

Another aspect of the invention provides a method of treating to a cancer in a subject, comprising administering to the subject an effective amount of the isolated EV and/or the composition of the present invention.

The cancer may be any cancer to which the methods and compositions described herein may be of therapeutic benefit. In some embodiments, the disorder may be cancer. In some embodiments, the cancer may be breast cancer, including but not limited to, BRCA⁺ breast cancer, HER⁺ breast cancer, progesterone receptor (PR)⁺ breast cancer, triple-negative (BRCA⁻/HER⁻/PR⁻) breast cancer, lobular breast carcinoma, ductal breast carcinoma, breast adenocarcinoma, and/or metastatic breast cancer. In some embodiments, the cancer may be recurrence of cancer in another site other than the breast (e.g., the lungs, the head and neck, the liver, the kidneys, the lymph nodes, etc.), wherein the cancer originates from metastatic breast cancer.

In some embodiments, the isolated EV may stimulate an immune response in the subject against the exogenously introduced antigen thereof. In some embodiments, the isolated EV may stimulate neutralizing antibodies in the subject against the exogenously introduced antigen thereof. In some embodiments, the isolated EV may activate cytotoxic lymphocytes (CTLs) and/or tumor-infiltrating lymphocytes (TILs)) in the subject against the exogenously introduced antigen thereof.

In some embodiments, the methods of the present invention may further comprise co-administering a cancer therapy. The cancer therapy may be any standard-of-care cancer therapy, including but not limited to, e.g., radiation, chemotherapy, surgery, immunotherapy and the like. In some embodiments, the subject may have previously received a cancer therapy. In some embodiments, the cancer in the subject may have been resistant to the previous immunotherapy.

Immunotherapy relevant to this invention include, but are not limited to, an autologous cellular immunotherapy, e.g., chimeric antigen receptor (CAR)-T cell therapy, CAR-NK cell therapy, and/or other modified immune cell (e.g., dendritic cell based therapy, e.g., Sipuleucel-T and the like)). In some embodiments, the immunotherapy may be targeted antibody therapy (e.g., monoclonal antibody therapy) such as, but not limited to, anti-CD20, anti-EGFR, anti-VEGF, anti-VEGFR2, anti-TNFα, anti-CD44, anti-CD19, anti-CD3, anti-EpCAM, anti-IGF1R, anti-MUC1, anti-CD51, anti-integrin, or any other targeted antibody-based therapy with anti-cancer function. In some embodiments, the immunotherapy may be immune checkpoint inhibitor therapy (“ICI”). In some embodiments, the immune checkpoint inhibitor therapy may be one or more inhibitor targeting CTLA-4, PD-1 and/or PD-L1. In some embodiments, the immune checkpoint inhibitor therapy may be, but is not limited to, pembrolizumab, nivolumab, ipilimumab, atezolizumab, avelumab, durvalumab, cemiplimab, amivantamab, apolizumab, bevacizumab, bivatuzumab, blinatumomab, camrelizumab, catumaxomab, cemiplimab, cixutumumab, clivatuzumab (e.g., clivatuzumab tetraxetan) durvalumab, edrecolomab, ertumaxomab, etaracizumab, faricimab, inebilizumab, intetumumab, isatuximab, margetuximab, necitumumab, nimotuzumab, Obinutuzumab, ocrelizumab, ofatumumab, olaratumab, panitumumab, pemtumomab, pertuzumab, racotumomab, ramucirumab, retifanlimab, rituximab, siltuximab, tafasitamab, teclistamab, tisotumab, tositumomab, trastuzumab, tremelimumab, votumumab and/or any variant or biosimilar thereof.

In some embodiments, the methods of the present invention may comprise co-administering the effective amount of the isolated EV and/or composition and another cancer therapy. In some embodiments, the methods of the present invention may comprise co-administering the effective amount of the isolated EV and/or composition and an immunotherapy. In some embodiments, the methods of the present invention may comprise co-administering the effective amount of the isolated EV and/or composition and an immune checkpoint inhibitor.

In some embodiments, co-administering the cancer therapy may comprise administering the therapy prior to, concurrently with, and/or after administering the effective amount of the isolated EV and/or composition.

In some embodiments, co-administering the cancer therapy concurrently with the isolated EV and/or composition may comprise administering the effective amount of the isolated EV and/or composition and the therapy as a single administration (e.g., in a single composition).

In some embodiments, co-administering the cancer therapy concurrently with the isolated EV and/or composition may comprise administering the effective amount of the isolated EV and/or composition and the therapy at about the same time (e.g., within about 1 minute to about 24 hours of each other, or any value or range therein), as two separate administrations.

In some embodiments, administering to the subject an effective amount of the isolated EV and/or composition may comprise administering a first dosage of the isolated EV and/or the composition and administering one or more (e.g., two or more, three or more, four or more, etc.) additional dosages of the isolated EV and/or the composition at least one, two, three, four, five, six, seven, eight, nine, ten, eleven, or twelve or more weeks after administering the first dosage.

Also provided herein are methods of producing the isolated EV and/or composition comprising the same of the present invention.

In some embodiments, a method of producing a nonnucleated extracellular vesicle (EV) comprising a surface membrane and comprising an interleukin-2 (IL-2) molecule and/or functional fragment thereof, a major histocompatibility (MHC) molecule and/or functional fragment thereof, and one or more costimulatory molecule, each expressed on the surface membrane of the EV, wherein the MHC molecule and/or functional fragment thereof presents an exogenously introduced antigen (e.g., the isolated EV of the present invention) is provided, the method comprising: (a) delivering to a culture of one or more antigen presenting cell that expresses major histocompatibility molecules I and II and which secretes nonnucleated EVs derived from the surface membrane of the APC (e.g., a macrophage, e.g., a dendritic cell), a IL-2 molecule and/or functional fragment thereof formulated to be expressed on the surface membrane of the APC, thereby producing an APC expressing the IL-2 molecule and/or functional fragment thereof on the surface membrane of the APC; (b) contacting (“culturing” and/or “stimulating”) the APC of step (a) with one or more innate immune activator, thereby inducing expression of one or more costimulatory molecules on the surface membrane of the APC; (c) introducing (“pulsing”; e.g., culturing) to the APC of step (b) a source of exogenous antigen (e.g., cancer cell lysate, e.g., isolated cancer cell RNA), thereby producing an APC expressing MHC-I and MHC-II, one or more costimulatory molecule and the IL-2 molecule and/or functional fragment thereof, on the surface membrane of the APC and on the surface membrane of the EVs secreted by the APC, wherein the MHC-I and/or MHC-II expressed on the surface membrane of the APC and the EVs presents the introduced exogenous antigen, thereby producing a nonnucleated EV comprising a surface membrane and expressing MHC-I and MHC-II, one or more costimulatory molecule and the IL-2 molecule and/or functional fragment thereof, on the surface membrane of the EV, wherein the MHC-I and/or MHC-II expressed on the surface membrane of the EV presents the introduced exogenous antigen.

In some embodiments, a method of producing an EV of the present invention may further comprise isolating the nonnucleated EV secreted from the APC of step (c); thereby producing the isolated nonnucleated extracellular vesicle comprising a surface membrane and expressing an interleukin-2 (IL-2) molecule and/or functional fragment thereof, a major histocompatibility (MHC) molecule and/or functional fragment thereof, and one or more costimulatory molecule, each expressed on the surface membrane of the EV, and wherein the MHC molecule and/or functional fragment thereof presents an exogenously introduced antigen.

The IL-2 molecule and/or functional fragment thereof may be delivered to the APC and/or the isolated EV through any suitable laboratory mechanism, as would be known to the skilled artisan. For example, in some embodiments, the IL-2 molecule and/or functional fragment thereof is delivered to the APC with a transfection agent (e.g., lipofectamine and the like) and/or transduction agent (a vector, e.g., a viral vector, e.g., lentiviral vector and the like).

In some embodiments, the IL-2 molecule and/or functional fragment thereof comprises a fusion protein of an APC-surface molecule linked to an IL-2 functional fragment. The term “APC-surface molecule” as used herein refers to any molecule commonly found membrane-bound (e.g., having a transmembrane binding portion) in an APC such as a mammalian macrophage and/or dendritic cell. APC-surface molecules of relevance to this invention include, but are not limited to, MFG-E8 or the like. In some embodiments, the IL-2 molecule and/or functional fragment thereof comprises a fusion protein of an MFG-E8 protein or transmembrane fragment thereof linked to an IL-2 functional fragment.

The innate immune activator molecule relevant to this invention may be any activator and/or agonist which stimulates the upregulation and expression of costimulatory molecules on a mammalian APC such as a macrophage and/or dendritic cell. In some embodiments, the innate immune activator molecule may be a cGAS-STING cytosolic DNA sensing pathway (“cGAS-STING” or “STING”) activator, and/or a Toll-like receptor (TLR) activator.

A STING agonist of the present invention may be any molecule which directly interacts (e.g., contacts and/or binds) STING and results in STING activation. In some embodiments, the STING agonist may be, but is not limited to a cyclic dinucleotide, e.g., cGAMP, c-diGMP, G10, DMXAA, diABZI, ADU-S100, MSA-2, or the like.

A TLR activator and/or agonist of the present invention may be any molecule which directly interacts (e.g., contacts and/or binds) a TLR and results in innate immune response activation. In some embodiments, the TLR activator and/or agonist may be, but is not limited to LPS, flagellin, or the like.

In some embodiments, the innate immune activator molecule may be a STING activator (e.g., cGAMP or the like) and a Toll-like receptor (TLR) activator (e.g., LPS). In some embodiments, the innate immune activator molecule may be a single molecule that is a STING activator (e.g., cGAMP or the like) and a Toll-like receptor (TLR) activator (e.g., LPS). In some embodiments, the innate immune activator molecule may be a more than one molecule (e.g., a combination of molecules; e.g., a mixture of molecules), wherein one or more is a STING activator (e.g., cGAMP or the like) and one or more is a Toll-like receptor (TLR) activator (e.g., LPS).

In some embodiments of the methods of the present invention, the method may further comprise providing the culture of one or more antigen presenting cell that expresses major histocompatibility molecules I and II and which secretes nonnucleated EVs derived from the surface membrane of the APC (e.g., a macrophage, e.g., a dendritic cell), wherein the providing comprises deriving the APC from a mammalian subject (e.g., a healthy subject, e.g., a patient).

In some embodiments, deriving the APC from a mammalian subject may comprise isolating the APC from sample from the subject. As used herein, the term “sample” is used in its broadest sense. In one sense, it is meant to include a specimen from a biological source.

A “sample” or “biological sample” of this invention can be any biological material, such as a biological fluid, an extract from a cell, an extracellular matrix isolated from a cell, a cell (in solution or bound to a solid support), a tissue, a tissue homogenate, and the like as are well known in the art. For example, biological samples can be obtained from animals (including humans) and encompass fluids (e.g., blood, mucus, urine, saliva), solids, tissues, cells, and gases. In some embodiments, the sample is obtained from a tumor (e.g., tumor stroma) in the subject. The sample may also comprise one or more immune cells, including T cells of the subject, including immune cells (e.g., helper T cells) from the tumor (e.g., tumor stroma) of the subject.

Also provided herein is a method of treating a cancer (e.g., breast cancer) in a subject in need thereof, comprising: (a) retrieving a sample from the subject, wherein the sample comprises an APC of the subject that expresses major histocompatibility molecules I and II and which secretes nonnucleated EVs derived from the surface membrane of the APC (e.g., a macrophage, e.g., a dendritic cell); (b) isolating one or more APC from the sample; (c) delivering to a culture comprising one or more APC isolated from the sample, a IL-2 molecule and/or functional fragment thereof formulated to be expressed on the surface membrane of the APC; (d) contacting (“culturing” and/or “stimulating”) the APC of step (c) with one or more innate immune activator, thereby inducing expression of one or more costimulatory molecules on the surface membrane of the APC; (e) introducing (“pulsing”; e.g., culturing) to the APC of step (d) a source of cancer antigen (e.g., cancer cell lysate, e.g., isolated cancer cell RNA) from the cancer of the subject, thereby producing an APC expressing MHC-I and MHC-II, one or more costimulatory molecule and the IL-2 molecule and/or functional fragment thereof, on the surface membrane of the APC and on the surface membrane of the EVs secreted by the APC, wherein the MHC-I and/or MHC-II expressed on the surface membrane of the APC and the EVs presents the introduced cancer antigen from the cancer of the subject, (f) isolating the EVs secreted from the APC of step (e); and (g) administering to the subject an effective amount of the isolated EVs and/or a composition comprising the same, thereby treating the cancer in the subject in need thereof.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1: IL2-ep13nsEV Prepared from DC were Educated with Tumor Lysate

Engineering of membrane-bound IL2 and induction of co-stimulatory factors on p13nsEV. To develop an active immunotherapy that can induce a specific anti-tumor effect through activation of lymphocytes even in the immune-suppressive environment, we decided to engineer the natural nanovesicle secreted by DC. Like DC cells, DC-derived sEVs retain essential functions to induce immunity. We isolated sEVs from the mouse DC cell line DC2.4 as well as human primary DC, after they were pulse-educated with tumor cell lysate. These nanovesicles were termed p13nsEV in this study. Following the guidance of Minimal Information for Studies of Extracellular Vesicles (Thery et al. 2018 J. Extracellular Vesicles 7:1535750), p13nsEV was verified to express enriched transmembrane protein CD63 and cytosolic protein TSG101. Low expression of AFOB indicates minimal contamination of lipoproteins. An ELISA was also performed on the purified p13nsEV to make sure that no residual stimulators used for differentiation and stimulation of DC were present. Next, p13nsEV were visualized by negative staining followed by electron microscopy. We found that these vesicles were approximately 100 nm in diameter with a saucer-like shape, consistent with the previous publications (Yang et al. 2013 Mol Med Rep 8:1272-1278). Importantly, the p13nsEV expressed the MHC I and MHC II molecules on the surface, as shown by immunostaining and EM. By performing nanoparticle tracking analysis (NTA), we found that the median diameter of p13nsEV was 114 nm. We also performed Western Blot to verify the expression of MHC I and MHC II in the same amount of membrane fraction of p13nsEV as well as the matured DCs that were pulsed with tumor cell lysate. It was found p13nsEV retained the antigen-presenting machinery MHC I and MHC II. Notably, more enrichment of MHC I and MHC II was observed in p13nsEV compared to DC.

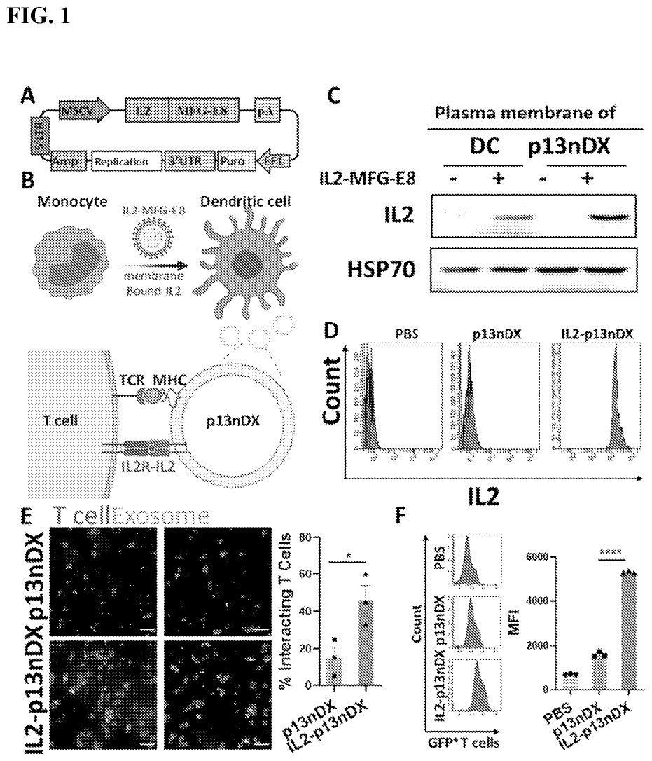

To examine the in vivo behavior of p13nsEV and test the possibility of using p13nsEV as an active immunotherapy to treat breast cancer, the p13nsEV from DC2.4-PalmtdTomato was harvested and injected into the tail base of C57BL/6 mice. After 6 hours, the inguinal LN (lymph node) and iliac LN were resected and visualized under the microscope, and p13nsEV was observed in the LNs. To further investigate the bio-distribution, p13nsEV was stained using ExoGlow-Vivo EV Labeling Kit (Near-IR, System Biosciences) followed by injecting them into mice intramuscularly through the tail base. At different time points, the mice were imaged using an IVIS® In Vivo Imaging System and the various organs were harvested to analyze the uptake of p13nsEV. We observed strong infiltration of p13nsEV into secondary lymphoid organs. These findings indicated that p13nsEV has the essential components of machinery for carrying neoantigens to immune cells and that intramuscular injection of p13nsEV makes the vesicle widely distributed into the circulation and other immune organs. Next, to construct an active immunotherapy that is capable of targeted induction of lymphocytes, we engineered it to express membrane-bound IL2 on the surface of the p13nsEV. IL2 is necessary for inducing functional memory CD8+ T cells following immunization (Laidlaw, Craft and Kaech 2016 Nature Reviews: Immunology 16:102-111) and its receptor is widely expressed in both T lymphocytes and B lymphocytes. By conjugating the IL2 on the surface of p13nsEV, we aimed to achieve the targeted delivery of p13nsEV to lymphocytes and enhance the IL2-induced stimulation of these immune cells. To accomplish this goal, MFG-E8, a protein that is known to be expressed on the sEVs from dendritic cells (Veron et al. 2005 Blood Cells Mol Dis 35:81-88), was utilized. The functional IL2 gene was fused with the C1C2 domain of MFG-E8 (FIG. 1 panel A), so that IL2 can be displayed on the surface of DC-derived sEVs. By the lentiviral expression of the IL2-MFG-E8 fusion protein in DCs, we generated IL2-p13nsEV that has both MHC-presented antigens and increased surface IL2 expression (FIG. 1 panel B). The membrane fraction of DCs and p13nsEV was extracted before and after the ectopic expression of IL2-MFG-E8. Western blot was performed to verify the successful localization of IL2 onto the membranes (FIG. 1 panel C). We also directly examined the surface IL2 on the IL2-p13nsEV by flow cytometry, and confirmed that IL2 is indeed upregulated after engineering IL2-MFG-E8 on DCs (FIG. 1 panel D). IL2 is a potent T cell growth factor, and its receptor is highly expressed by T lymphocytes and induces their proliferation. Therefore, by conjugating IL2 on the surface of p13nsEV, we aimed to generate an immunotherapy vesicle targeting T-lymphocytes. When IL2-p13nsEV was cultured with T lymphocytes, we indeed observed significantly increased interaction between the vesicles and T cells, compared to p13nsEV (FIG. 1 panel E). Flow cytometry was also performed to measure p13nsEV-positive T cells, and we found increased interaction with T cells in the IL2-p13nsEV group (FIG. 1 panel F).

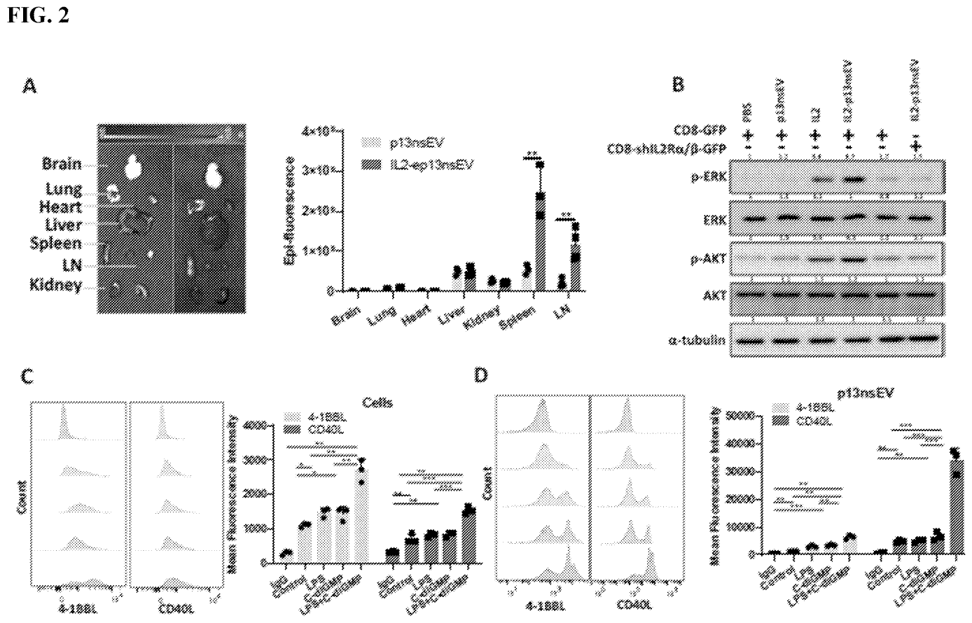

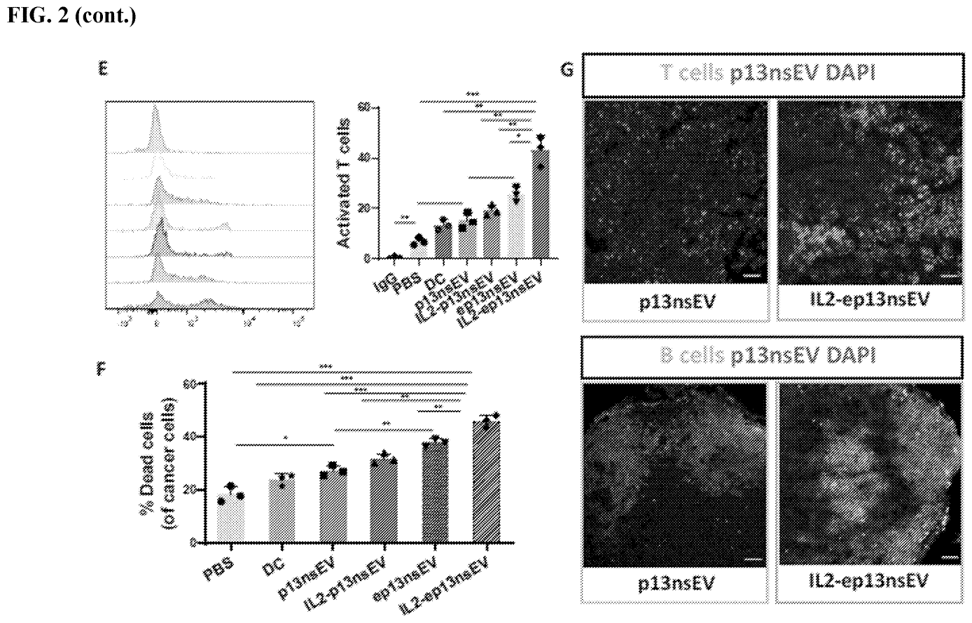

With the observation that the engineered IL2 on the surface of p13nsEV significantly increase T cell targeting ability (FIG. 1 panels E and F), we next tested the functional ability of the membrane IL2 in guiding p13nsEV to lymphocytes in vivo. p13nsEV was labeled with fluorescent dye using the ExoGlow-Vivo EV Labeling Kit. They were then administered into the C57BL/6 mice via tail base injection. After 6 hours, when p13nsEV reached the plateau of infiltration into the lymphoid organs, the mice were imaged using IVIS® system and the various organs were harvested to analyze the uptake of p13nsEV. We observed a strong increase in vesicle infiltration into secondary lymphoid organs with IL2 expressed on the surface of p13nsEV (FIG. 2 panel A). Thus, this result further confirmed that membrane IL2 was capable of guiding p13nsEV to lymphocytes. Next, we tested if the membrane IL2 was able to be functional to activate the IL2 receptor after it brings the vesicle to the T cells. The p13nsEV with or without the surface IL2 was incubated with T cells, and the result of Western blot indicates that the surface IL2 is functional as IL2-p13nsEV was able to induce downstream signaling. But p13nsEV failed to do that. The induction of the signaling was through the IL2 receptor on T cells as the IL2 antagonist, as well as knockdown IL2 receptor α and β (IL2Rα/β), blocked the phosphorylation of the ERK and AKT (FIG. 2 panel B). In addition to cytokines, co-stimulatory factors are also essential for T cell activation (Chen and Flies 2013 Nature Reviews: Immunology 13:227-242). The STING agonist was previously found to induce dendritic cell activation and maturation (Marinho et a. 2018 J Innate Immun 10:239-252). Accordingly, we compared the effect of LPS and STING agonist C-diGMP on the expression of co-stimulatory factors after ex-vivo dendritic cell differentiation from monocyte by GM-CSF. We observed a substantial effect of LPS and STING agonist on co-stimulatory factor expression when monocyte was treated with both reagents, while LPS or STING agonist alone was not able to induce this change. In DCs, the levels of mRNA 4-1BBL, CD40L, and Tim3 were significantly increased after the LPS and STING agonist treatment. Next, we verified the expression of these co-stimulatory factors on the surface of DCs after treatment using LPS and STING agonist. This induction by the combination treatment was verified in 4-1BBL and CD40L (FIG. 2 panel C). Consistent with these results, 4-1BBL and CD40L proteins were also increased on the surface of IL2-p13nsEV after the LPS and STING agonist treatment (FIG. 2 panel D). The Tim3 expression on the surface of DCs was also increased after LPS or C-diGMP treatment. However, only negligible increase of its expression was observed on IL2-p13nsEV. Furthermore, no synergy was observed in the combination treatment. 4-1BBL has been reported to be the most effective signaling molecule for activating lymphocytes (Martinez-Perez et al. 2021 International Journal of Molecular Sciences 22), and CD40L is known to maintain full activation of T cells. Accordingly, we hypothesized that this p13nsEV which expresses co-stimulatory factors induced by LPS/STING agonist combination treatment will have an enhanced ability to activate T cells. Thus, in this study it was named the enhanced p13nsEV (ep13nsEV). Because IL2-ep13nsEV contains the MHC-bound antigen that is capable of priming T cells, as well as the surface IL2 and co-stimulatory factors known to assist T cell activation, we theorized that this active immunotherapy would be capable of inducing tumor cell-killing immunity independent of host APC cells. Indeed, this hypothesis was verified by the T cell activation assay. The IL2-ep13nsEV was generated from ovalbumin (OVA)-pulsed DCs that were treated with lentivirus expressing membrane IL2 or C-diGMP, or both. Then they were used to prime the CD8 T cells isolated from the OT-I mice in vitro. CD8 T cells from the OT-I mice specifically express the T cell receptor of OVA. Notably, IL2-ep13nsEV induced the highest level of CD8 T cell activation compared to other groups (FIG. 2 panel E). It also induced increased activation of the antigen specific T cells compared to the DC vaccine, which was pulsed with OVA protein. These primed CD8 T cells from OT-I mice were also tested in the cytotoxicity assay with cancer cells expressing OVA. We found that membrane IL2 alone induced only negligible cytotoxicity in T cells. However, co-stimulatory factors of ep13nsEV and IL2-ep13nsEV, with both IL2 and co-stimulatory factors, induced significant cytotoxicity in the primed T cells against cancer cells (FIG. 2 panel F). When administered in vivo, IL2-ep13nsEV showed increased infiltration into the T cell and B cell loci in the LN (FIG. 2 panel G). After injection of Vybrant DiD labeled sEVs, an increased amount of T cell interacting IL2-ep13nsEV was verified compared to p13nsEV. These results indicate an autonomous functionality of membrane IL2 of IL2-ep13nsEV in lymphocyte seeking, while the co-stimulatory factors of IL20ep13nsEV have a strong potency in T cell activation. Thus, the membrane IL2 and co-stimulatory factors of IL2-ep13nsEV could assist the T cells priming and enhance the antigen presentation.

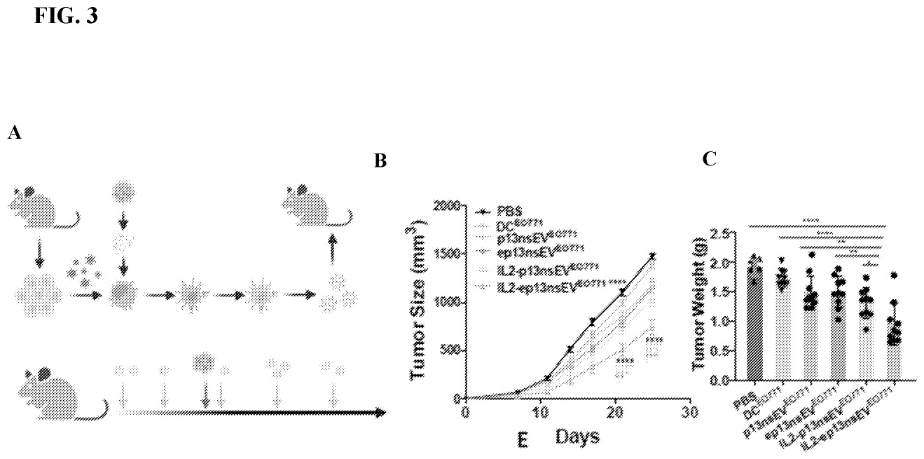

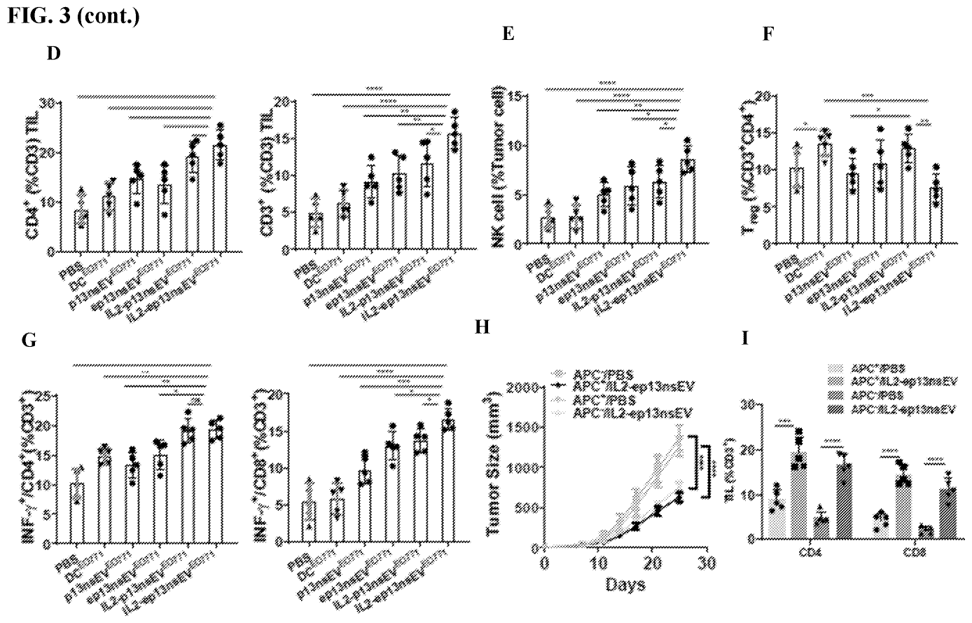

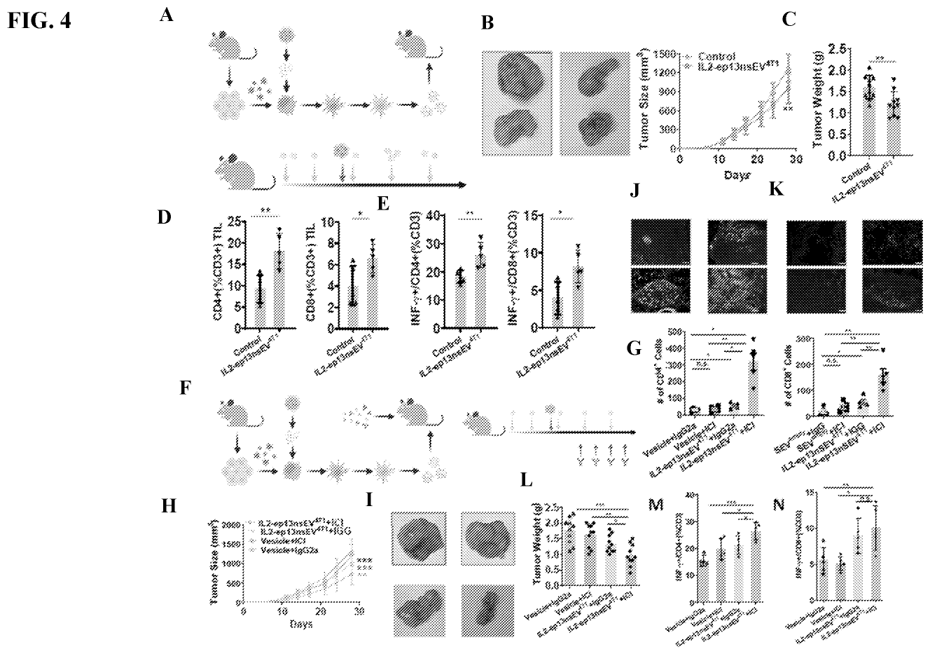

Effects of IL2-ep13nsEV on breast tumor growth in a syngeneic mouse model. Next, we tested if IL2-ep13nsEV was able to enhance the cancer cell-specific killing of cytotoxic T lymphocytes in vivo. DCs were infected with lentivirus expressing the IL2-MFG-E8 fusion protein, pulse-educated with EO771 tumor cell lysate, and matured with LPS and C-diGMP. IL2-ep13nsEVE0771 was then purified through sequential ultracentrifugation and used to treat the syngeneic mice implanted with EO771 cells (FIG. 3 panel A). The tumor growth in the mice treated with IL2-ep13nsEVEO771 was tracked, and control groups were treated with PBS or DC vaccine loaded with tumor lysate, or other sEVs, noting that PBS and empty p13nsEV did not affect the tumor growth at all. IL2-ep13nsEVEO771 resulted in significantly slower tumor growth compared to the control groups (FIG. 3 panel B). After 4 weeks of tumor growth, IL2-ep13nsEVEO771 treatment led to lowest tumor burdens in mice (FIG. 3 panel C). Next, we investigated if IL2-ep13nsEVEO771 could mobilize T cells into tumor lesions. The tumors were dissociated and both of CD4 and CD8 TIL (tumor-infiltrating lymphocytes) were measured by flow cytometry (FIG. 3 panel D). The increase in both populations was observed in the tumors treated by IL2-ep13nsEVEO771. Furthermore, an increase of NK cells was also observed in the mice treated with IL2-ep13nsEVEO771 (FIG. 3 panel E). There was no significant difference in the population of regulatory T cells (Tregs) between the p13nsEVEO771 treatment and IL2-ep13nsEVEO771 treatment, while IL2-ep13nsEVEO771 decreased the Tregs compared to PBS and IL2-p13nsEV (FIG. 3 panel F). IL2-ep13nsEVEO771 also induced more activated CD4 and CD8 T cells in the spleens of the mice (FIG. 3 panel G). To further verify the antigen presenting function of IL2-ep13nsEV, the DC, B cells and macrophages were depleted in the CD11C-DTR mice by injection of diphtheria toxin, anti-CD20 and Clophosome before IL2-ep13nsEV treatment. In the APC-depleted mice, IL2-ep13nsEV was able to suppress the tumor growth at comparable level as in the APC intact mice (FIG. 3 panel H), and induce both CD4 and CD8 T cells (FIG. 3 panel I). These results supported our hypothesis that IL2-ep13nsEV could perform antigen presentation in replacement of APC. EO771 syngeneic breast carcinoma model was found to have a favorable response to immunotherapies (Le Naour, Rossary and Vasson 2020 Cancer Med 9:8074-8085). To model a breast carcinoma with enhanced Immune evasion, we also tested our active immunotherapy in the 4T1 breast cancer cells (FIG. 4 panel A). 4T1 breast cancer cells were previously found to be immune-cold (Wu et al. 2010 iScience 23:101341), and it was reported to have a low mutation burden and weak T cell infiltration (Yang et al. 2017 Oncotarget 8:30621-30643). Resistance to immunotherapies was observed in the 4T1 syngeneic mouse model (Xie et al. 2018 J Immunother Cancer 6:88). In this study, IL2-ep13nsEV4T1 was found to significantly suppress tumor growth compared to the control group (FIG. 4 panels B and C). Consistent with this finding, a significant increase in the TIL (FIG. 4 panel D) and activated splenocyte (FIG. 4 panel E) were observed in the mice treated with IL2-ep13nsEV4T1 compared to the PBS control group, noting PBS and empty p13nsEV did not affect the 4T1 tumor growth as well. This tumor-suppressive effect of IL2-ep13nsEV4T1 relies on the presence of IL2-MFG-E8 fusion protein and the increased the co-stimulatory factors on the active immunotherapy vesicles. The p13nsEV4T1, the vesicle without the engineered IL2 and enhanced co-stimulatory factors on the membrane, was not able to suppress the tumor growth in the immune-cold 4T1 breast cancer in the syngeneic mice. The inclusion of IL2 or co-stimulatory factor could not achieve any significant decrease in tumor size. Currently, adoptive DC cell transfer is being extensively studied as another approach of active immunotherapy in more than 20 breast cancer-related clinical trials (Fuentes-Antras et al. 2020 Front Oncol 10:605633). Notably, DC4T1, the DC-based adoptive cell transfer could not suppress the 4T1 tumor growth. This result emphasizes the significance of IL2-ep13nsEV as an active immunotherapy for breast cancer. To elucidate the importance of CD4 and CD8 T cells in IL2-ep13nsEV-mediated tumor cell suppressive effect, the CD4 or CD8 cells were depleted before the treatment with IL2-ep13nsEV. CD4 and CD8 were both found to be essential for IL2-ep13nsEV4T1 to execute its suppressive effect against cancer cells as depletion of either of them resulted in compromised effect of IL2-ep13nsEV4T1.

IL2-ep13nsEV potentiates ICI in treating breast cancer. Considering the capability of IL2-ep13nsEV4T1 in promoting antigen presentation, we hypothesized that this active immunotherapy could benefit the efficacy of ICIs as they theoretically complement each other in rallying reactive immune response. When IL2-ep13nsEV promotes the generation of more cancer cell-specific lymphocytes, the ICIs could keep them viable, so that these activated lymphocytes could more effectively eliminate the cancer cells. Accordingly, we tested the efficacy of the combination of IL2-ep13nsEV4T1 and anti-mouse PD1 in vivo. In addition to the treatment with IL2-ep13nsEV4T1 treatment, ICI was also administered four times to the mice implanted with 4T1 cells that are known to be resistant to ICI (FIG. 4 panels F and G). While ICI alone was not able to decrease tumor size, the combination of IL2-ep13nsEV4T1 and ICI sensitized the tumor cells to ICI as ICI further decreased tumor growth on top of the suppressive effect of IL2-ep13nsEV4T1 (FIG. 4 panels H and I). The combination of both reagents also significantly increased the number of TIL (FIG. 4 panels J and K) and activated T cells in spleen (FIG. 4 panels L and M) compared to the mono-treatment of IL2-ep13nsEV4T1 or ICI.

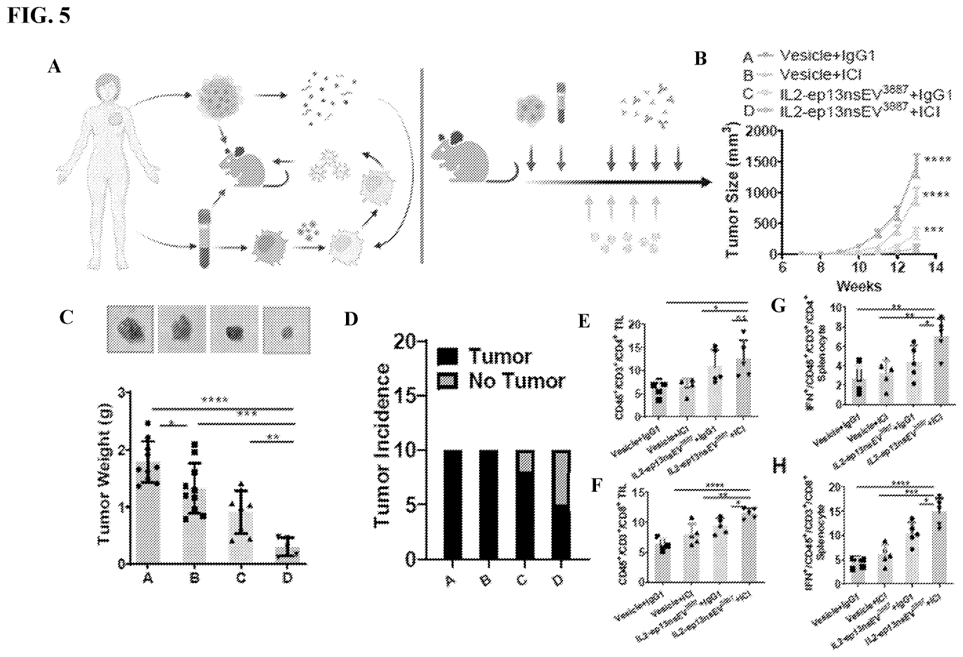

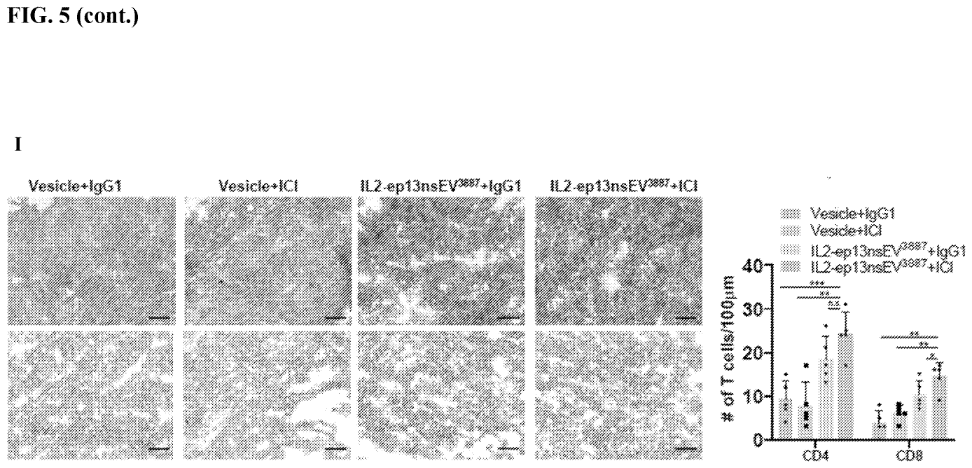

To verify our results in a more clinically relevant model, we established humanized PDX to test the anti-tumor effect of IL2-ep13nsEV. PDX3387 was verified for its expression of PD-L1 for rationalizing the combination use of ICI with IL2-ep13nsEV. Haplotyping was performed to identify haploidentical PBMC for the construction of the humanized mice. The successful construction of humanized PDX was verified by the growth of human CD45 cells in the peripheral blood and the infiltration of lymphocytes into the primary PDX tumor. Next, the humanized PDX mice were treated with IL2-ep13nsEV3887 that were prepared from IL2 engineered and co-stimulatory factors induced DCs, with or without ICI (FIG. 5 panel A). The mono-treatment of IL2-ep13nsEV3887 or ICI alone was found to be able to suppress the PDX tumor growth in the humanized mice. But the combination of both reagents inhibited the tumor growth with a significantly greater efficacy (FIG. 5 panels B and C). More importantly, while IL2-ep13nsEV3887 or ICI alone was not able to affect tumor incidence, the combination of both reagents resulted in a reduction of tumor incidence by 50% (FIG. 5 panel D). Consistent with the finding in the 4T1 model, IL2-ep13nsEV3887 was also able to induce both CD4/CD8 TIL. The result of flow cytometry analyses presented increased hCD45+/hCD3+/hCD4+ and hCD45+/hCD3+/hCD8+ TIL (FIG. 5 panels E and F). The increase in activated CD4/CD8 splenocytes was also observed in the mice treated with IL2-ep13nsEV3887 (FIG. 5 panels G and H). IHC for the CD4 and CD8 cells also suggests that the active immunotherapy vesicle treatment promoted an increased amount of T lymphocytes infiltration into the tumor (FIG. 5 panel I).

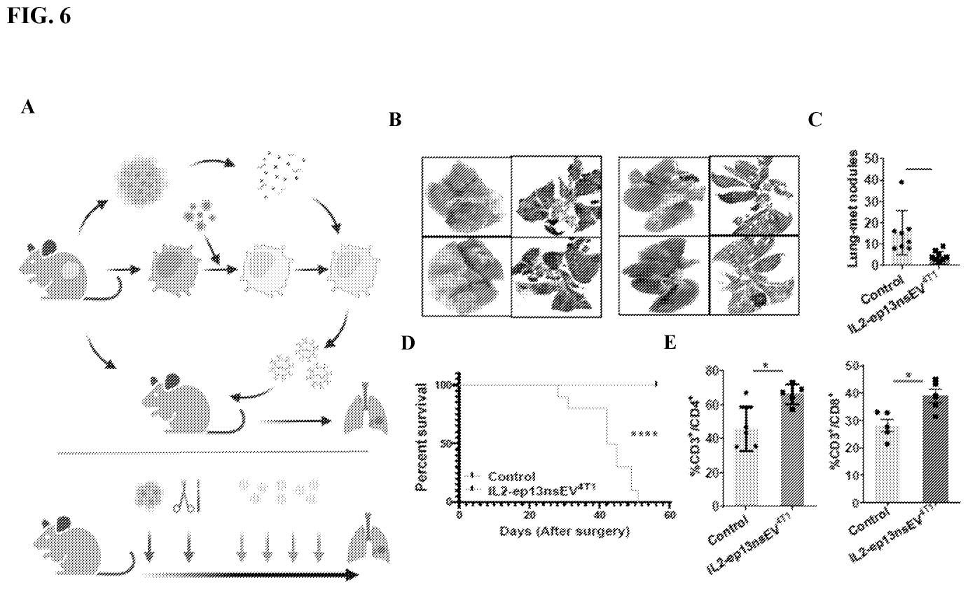

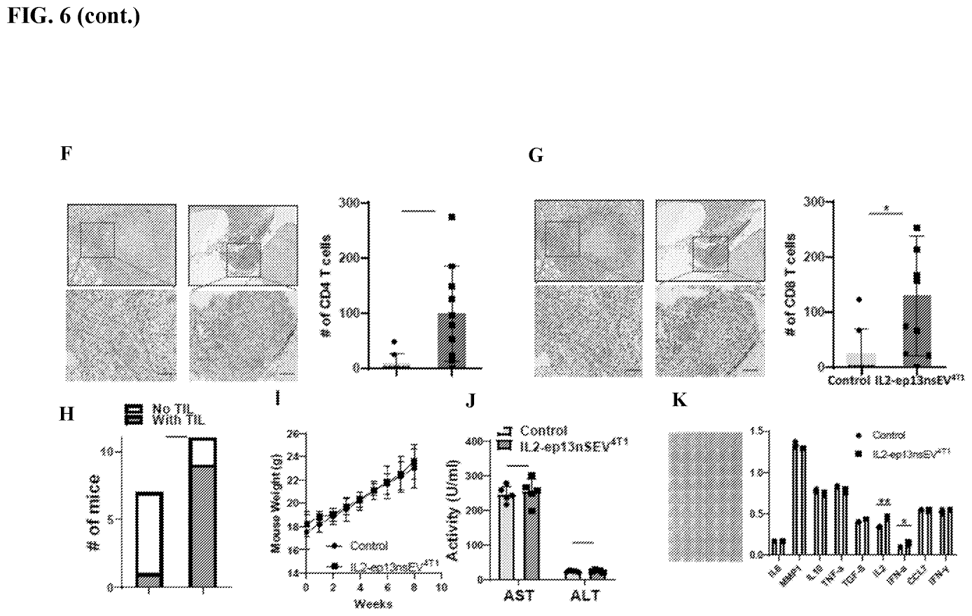

The use of IL2-ep13nsEV to prevent tumor recurrence. While the ICI treatment is primarily given to breast cancer patients at advanced stages, surgery is still the most curable approach for patients at an early stage. However, recurrence is often observed in many patients, which is the primary cause of breast cancer-related death. For these patients, with resected tumor tissue available, it is feasible to generate IL2-ep13nsEV as personalized active immunotherapy to prevent future recurrence. Therefore, we explored if such treatment could be used to prevent the relapse of tumors in a syngeneic surgical recurrence model (FIG. 6 panel A). One week after implantation of 4T1 tumor, the mice received surgical treatment to remove the primary tumor. The resected tumor was used to generate the lysate and pulsed into mouse dendritic cells with engineered IL2 and induced co-stimulatory factors on the surface. IL2-ep13nsEV4T1 was then purified and injected into mice on day 3 after the surgery. After five weeks, the lungs of these mice were examined for recurrence (FIG. 6 panel A). We found that this personalized treatment significantly decreased the number of recurrent lung metastatic nodules (FIG. 6 panels B and C). Furthermore, in another two groups of mice that were tracked for survival under treatment, IL2-ep13nsEV4T1 significantly extended the survival of the mice after primary surgery (FIG. 6 panel D). There was an increase of the lymphocytes in the blood after the treatment with the engineered active immunotherapy (FIG. 6 panel E). Staining the metastatic lesions in the lung for CD4 and CD8 positive cells revealed an increased number of both populations of lymphocytes in the IL2-ep13nsEV4T1 treated group (FIG. 6 panels F and G). Furthermore, more mice were found with the presence of TIL in the metastatic lesions in the IL2-ep13nsEV4T1 treated group compared to the control group (FIG. 6 panel H). Considering the fact that recurrence of breast cancer tends to be late-onset (Takeshita et al. 2019 Scientific Reports 9:16942), the preventive use of the engineered active immunotherapy should be long-lasting and non-toxic. We looked at the potential side-effects of IL2-ep13nsEV4T1 and found that there was no change in body weight for 8 weeks (FIG. 6 panel I). The blood of mice after 30 days of IL2-ep13nsEV4T1 treatment was analyzed for liver function (FIG. 6 panel J) and no change of AST and ALT was found. A profiling of inflammatory cytokines revealed that there was no sign of cytokine release syndrome or immune-cell hyperactivation associated with IL2-ep13nsEV4T1 (FIG. 6 panel K).

### Example 2: Methods as Used Herein in Examples 1 and 3

Cells and Cell Culture—Mouse breast carcinoma cell lines, 4T1 and EO771 were purchased from American Type Tissue Culture Collection (ATCC). 4T1 cells were cultured in RPMI-1640 Medium (Invitrogen) with 10% of FBS (fetal bovine serum). EO771 cells were cultured in DMEM (Dulbecco's Modified Eagle's Medium, Invitrogen) with 10% FBS and 20 mM HEPES. Mouse dendritic cell line DC2.4 was purchased from Sigma-Aldrich and cultured in RPMI-1640, 10% FBS, 1×L-Glutamine, 1× non-essential amino acids, 1×HEPES Buffer Solution. Murine bone marrow-derived derived dendritic cells (BMDCs) were generated as previously described (26, 27). Briefly, bone marrow cells were collected by flushing the femur and tibia bones of 6 to 8-week-old C57BL/6 mice (The Jackson Laboratory) with ice-cold RPMI 1640 Medium. After collection, the cells were washed, then incubated in RPMI-1640 with 10% FBS and 20 ng/ml GM-CSF for 6 days to separate the non-adherent and adherent cells. The floating cells and loosely adherent cells were collected and the immature BMDCs were further purified by depleting F4/80+ macrophages with anti-F4/80 MicroBeads (Miltenyi Biotec). The immature BMDCs were educated with the tumor lysate for 24 hours followed by treatment with LPS (100 ng/ml) and c-di-GMP (200 μM). Human DCs were generated from human primary PBMC (HumanCellsBio) according to the previous publication (28, 29). Briefly, human PBMC was cultured in RPMI-1640 with 500 UI/ml of recombinant human GM-CSF and 300 UI/ml of recombinant human IL-4 (PeproTech Inc.). The differentiated DCs were collected and pulsed with tumor lysate for 24 hrs, followed by DC maturation by treating them with LPS (100 ng/ml) with or without c-di-GMP (200 μM)(30).

Engineering of p13nsEV—To engineer the IL2 expression on the surface of p13nsEV, a lentiviral plasmid coding the IL2-MFG-E8 fusion protein was used. Lentivirus was produced by transfecting 800 ng lentiviral plasmid, 600 ng viral packaging plasmid pPAX2, and 200 ng envelope plasmid pMD2.G into HEK293T cells. Primary monocytes and DC2.4 cells were infected and selected with 2 μg/ml puromycin (ThermoScientific). After virus infection, the monocytes and DC2.4 were differentiated and pulsed with tumor lysate as described above. The p13nsEV was isolated from the mature DCs by sequential ultracentrifugation.

Animal experiments—Experimental protocols were approved by the Institutional Animal Care and Use Committee at Wake Forest Health Science. For the 4T1 syngeneic breast cancer mouse model, 1.0×104 4T1 cells were injected with Matrigel® Matrix High Concentration (Corning, USA) into the mammary fat pads of 6 to 8-week-old female BALB/c mice (The Jackson Laboratory). For the syngeneic EO771 mouse breast cancer model, 1.0×105 cancer cells were injected with Matrigel® Matrix High Concentration into the mammary fat pads of 6 to 8-week-old female C57BL/6 mice. To investigate the effect of active immunotherapies and ICIs on the primary tumor of the syngeneic mice, IL2-ep13nsEV was given to the mice by intramuscular injection into the tail base at 50 μg each time on 3 and 7 days before tumor cell implantation, 1, 8, 15 days after tumor cell implantation. The anti-PD1 (Bio X Cell) was given to the mice at 20 mg/kg by i.p. (intraperitoneal) injection on day 7, 11, 15, 19 post tumor implantation. The growth of tumors was monitored and quantified by measuring the length and width of a tumor using an electronic caliper. Tumor volume was calculated by the modified ellipsoidal formula: volume=1/2 (length×width2). To study the antigen presenting function of IL2-ep13nsEV, the CD11c-DTR mice (The Jackson Laboratory) were injected with diphtheria toxin (i.p., 2 ng/g of body weight for DC depletion), anti-CD20 (i.p., 100 μg for B cell depletion) and Clophosome (intravenously, 8.5 μg/g of body weight for macrophage depletion) 24 hours before IL2-ep13nsEV treatment. The mice in APC+ group received injection of IgG2c isotype control and empty liposome. The IL2-ep13nsEV was given at the same dose and frequency as in the EO771 syngeneic mouse model. To deplete CD4 and CD8 T cells, mice were administered with anti-CD8-α or anti-CD4 antibodies (Bio X Cell) through i.p. injection at the dose of 150 μg/mouse on day 3 and 7 before tumor cell implantation. For in vivo p13nsEV uptake assay, the mice were treated with 50 μg Vybrant DiD-labeled p13nsEV or ExoGlow™-labeled p13nsEV. After 3, 6, 12, 18, 24 hours, the organs were extracted and imaged by using IVIS Xenogen bioimager (Caliper Life Science). To construct the tumor recurrence mouse model, 1.0×104 4T1 cells were injected into the mammary fat pads of 6 to 8-week-old female BALB/c mice. After one week, the primary tumors were surgically removed. The mice were treated with p13nsEV for 4 weeks on day 3, 10, 17 and 24 post surgery. At the endpoint, the lungs were collected for examination of recurrence. To construct the humanized PDX mice, the haplotyping was performed using LinkSeq™ HLA-ABDR typing kit (One Lambda). Next, 100×106 dissociated PDX tumor cells were implanted into the mammary fat pads of the humanized mice. The next week, 10×106 human PBMC were injected into the lateral tail vein of NOD/Lt-scid IL2r null mice as indicated by a previously published protocol (37). The sEV treatment was given at week 3, 5, 7, and 9 post PDX implantation. The ICI treatment was given at 20 mg/kg by i.p. at week 5, 7, 9 and 11 post tumor implantation.

Purification and Tracking Analysis of sEVs—sEVs from cells were isolated by ultracentrifugation. Cells were grown in SEV-depleted media for 48 h, and CM was collected for sEVs purification. SEVs were isolated by differential centrifugation as described before (32, 33). Briefly, the CM was centrifuged at 300 g for 10 minutes to remove cells. Cell debris was then removed by centrifugation at 2,000 g for 20 minutes. The supernatant was centrifuged at 16,500 g for 20 minutes to remove microvesicles. To remove the particles with the diameter larger than 200 nm, the supernatant was passed through a 0.2 μm filter (Sarstedt). SEVs were then pelleted by ultracentrifugation at 120,000 g for 70 minutes. The isolated sEVs were analyzed and quantified by nanoparticle tracking analysis (NTA) and electron microscope with negative staining.

Analysis of sEVs by Transmission Electron Microscopy—For negative staining, purified sEVs were placed on the discharged 200 mesh copper EM grids and fixed with 2% Paraformaldehyde (PFA). 1% uranyl acetate solution (Electron Microscopy Sciences) was used to stain the sEVs on the EM grids. The stained sEVs were then imaged by FEI Tecnai BioTwin Transmission Electron Microscope. For immunostaining of MHC I and MHC II on the surface of sEVs, Anti-MHC I (1:100) (Santa Cruz Biotechnology, sc-59199) and anti-MHC II (1:100) (Santa Cruz Biotechnology, sc-59318) were used to treat the fixed sEVs on the grids. The 10 nm colloidal gold-labeled protein G (1:50)(BOSTER BIOLOGICAL TECHNOLOGY, Pleasanton, CA, USA) was then used to label the primary antibodies. Negative staining was performed after immunostaining and the grid was imaged by transmission electron microscopy.

T cell-p13nsEV interaction assay—DC2.4 cells were labeled with PalmGFP encoded by lentivirus. Cells with intense fluorescence were selected by WOLF Cell Sorter. The p13nsEV labeled with PalmGFP were isolated by differential ultracentrifuge. To monitor the interaction between p13nsEV and T cells, Vybrant DiD-labeled T cells were seeded in chamber slides and 20 μg/ml p13nsEV were added to the media. After 24 hours of incubation, the cells were washed and fixed, followed by sealing the slides with coverslips. The p13nsEV interacting with T cells was examined by observing the GFP signal under Keyence All-in-one Fluorescence Microscope (BZ-X700).

T cells activation and T cell cytotoxicity assay ex vivo after p13nsEV treatment-Pan-T cells (130-095-130, Miltenyi Biotec) or CD8 T cells (130-104-075, Miltenyi Biotec) were isolated from mouse spleen. T cells were primed by different groups of p13nsEV treatment for 5 days. After priming, the p13nsEV was washed away and T cells were co-cultured with tumor cells at 10:1 (tumor cell:T cell) for two days before being tested for IFN-y expression by flow cytometry. For the T cell cytotoxicity assay, the p13nsEV treated T cells were then co-cultured with 50 μg Vybrant DiD-labeled cancer cells, which is the same source of tumor lysate to generate the p13nsEV. After 5 days of co-culture, the death of cancer cells was examined by staining cells using the Zombie Aqua™ Fixable Viability Kit (BioLegend) followed by flow cytometry.

Flow cytometry—The cultured tumor cells were detached with Trypsin-EDTA (0.25%) solution and were washed twice with PBS. The cells were then stained with Zombie Aqua Fixable Viability Kit. For detecting the activated T cells in culture, the cells were treated with 2.5 mg/ml Brefeldin A (BioLegend) at 37° C. for 4 h before being washed twice with PBS. Then the cells were blocked with anti-CD16/32 (BioLegend), washed twice, and fixed in 4% PFA in PBS for 20 min. Intracellular staining was done by treating them with the permeabilization buffer (BioLegend). Cells were then stained with antibodies for 30 min. For immunophenotyping of tumors, spleens, and blood, tissue samples were dissociated and red blood cells were depleted using RBC Lysis Buffer (BioLegend). The cells were then treated with 2.5 mg/ml Brefeldin A at 37 ° C. for 4 h if intracellular cytokine staining will be performed. Fc-blocked was performed using Human TruStain FcX™ or Mouse TruStain FcX™ PLUS (BioLegend). Cells were then incubated with the antibodies diluted in staining buffer (2% FBS in PBS) at 4° C. in the dark for 20 min. After that, dead cells were stained using Zombie Aqua Fixable Viability Kit (BioLegend). The cells were then washed twice and analyzed or further fixed in 4% PFA in PBS for 20 min for intracellular cytokine staining. Intracellular staining was done by treating the cells with the permeabilization buffer (BioLegend). Cells were then stained with antibodies for 30 min. After staining, cells were examined by BD CantoII Flow Cytometer and the data was analyzed by FlowJo software. We gated TIL by ZombieDye-CD3+/CD4+ or CD8+, Natural killer (NK) cells by ZombieDye-CD3−/NK1.1+ and activated lymphocyte by ZombieDye-CD3+/CD4+ or CD8+/IFN-γ+, and regulatory T cells by ZombieDye-CD25+/CD4+/Foxp3+.

Immunohistochemistry (IHC)—IHC was carried out for surgically resected, paraffin-embedded specimens. Briefly, the sections were de-paraffinized in xylene, rehydrated, and heated at 95° C. for 30 min in sodium citrate buffer for antigen retrieval. They were treated with peroxidase blocking reagent (3% H2O2) for 15 min, followed by blocking with 5% BSA solution for 30 min. After blocking, the slides were incubated with primary antibodies targeting CD4 (Invitrogen, 14-0049-80) and CD8 (Invitrogen, MA1-80231) for 16 h at 4° C. After washing with PBS/0.1% Tween-20, the sections were treated with the secondary antibody anti-rabbit (Bio-Rad). The sections were washed 3 times, and DAB substrate chromogen solution was used to develop the signal of target proteins, followed by counterstaining with hematoxylin. For negative control, we used the rabbit IgG isotype control (Invitrogen) instead of primary antibodies. The stained sections were randomized. The amount of the CD4 and CD8 T cells was evaluated by concordance among the counting of three independent reviewers.

ALT/AST Activity Assay—The ALT (alanine aminotransferase) activity was detected using ALT Activity Assay kit (Sigma-Aldrich, USA) according to the product information sheet. Briefly, 100 μL of the Master Reaction Mix was added to each of the standard, positive control, and test samples in each well of 96-well plates. After 2-3 minutes (Tinitial), take the initial measurement of absorbance at 570 nm (A570) initial. The plate was then incubated at 37° C. protected from light, and measurements were taken every 5 minutes until the highest value of the samples is greater than the highest value of the standards. The penultimate reading (Tfinal) was the final measurement for calculating the enzyme activity (A570) final. To calculate the ALT activity, the background was corrected by subtracting the value obtained for the blank standard from all standard readings. The amount of pyruvate generated (B) between Tinitial and Tfinal was calculated by plotting the ΔA570=(A570) final−(A570) initial to the to the pyruvate standard curve. The ALT Activity=B×Sample Dilution Factor/((Tfinal−Tinitial)×Sample volume). AST (aspartate aminotransferase activity was measured with AST Activity Assay kit (Sigma-Aldrich, USA) according to product information sheet. Briefly, 100 μL of the Reaction Mix was added to each of the well. After 2-3 minutes (Tinitial) incubation at 37° C., initial measurement of absorbance at 450 nm (A450) initial was taken. The measurements were then taken every 5 minutes until the value of the most active sample is greater than the value of the highest standard. The penultimate reading (Tfinal) was the final measurement for calculating the enzyme activity (A450) final. To calculate the ALT activity, background was corrected by subtracting the value obtained for the blank standard from all standard readings. The amount of glutamate generated (B) between Tinitial and Tfinal was calculated by plotting the ΔA450=(A570) final−(A570) initial to the glutamate standard curve. The AST Activity=B×Sample Dilution Factor/((Tfinal−Tinitial)×Sample volume).

Real-Time PCR and Western blotting—Total RNA was isolated by using Direct-zol RNA MiniPrep Plus (Zymo Research Corp). Then iScript Reverse Transcription Supermix (Life Science Research) was used for reverse transcription. Quantitative PCR (qPCR) was conducted by using iTaq Universal SYBR Green Supermix (Life Science Research). Primers used to perform the qPCR were listed in Supplementary Data 1. Western blotting was carried out using a standard method. Briefly, the total protein of cells was extracted using RIPA buffer with Halt™ Protease Inhibitor Cocktail (Thermo Scientific). The plasma membrane protein was extracted by Plasma Membrane Protein Extraction Kit (Abcam, USA). The protein concentration was measured by Protein Assay (Bio-Rad, USA). Next, same amount of proteins were resolved by 10% SDS-polyacrylamide gel before being electrotransferred to nitrocellulose membrane. Primary antibodies against MHC I (Santa Cruz Biotechnology, sc-32235 for human and sc-59199 for mouse), MHC II (Santa Cruz Biotechnology, sc-59318), HSP70 (Invitrogen, Clone 5A5, #MA3-007), IL-2 (Cell Signaling Technology, #12239), Tsg101 (Invitrogen, Clone 4A10, MA1-23296), CD63 (Invitrogen, PA5-92370), APOB (Abcam, ab20737), TGF-β (Cell Signaling Technology, #3711) were used. The secondary antibodies used in this study include mouse IgG kappa binding protein m-IgGκ BP-HRP (Santa Cruz Biotechnology, sc-516102), HRP-conjugated anti-mouse IgG (Cell Signaling Technology, #7076) and anti-rabbit IgG (Bio-Rad, #1706515). Immunoblotting images were captured by Amersham Imager 600 or the X-ray film processor depending on availability.

Statistical tests and reproducibility Two tailed unpaired t-tests were used to compare the gene expression, cell population, and tumor size between two samples. Chi-squared test was used to compare the incidence of tumor growth and recurrence between different treatment groups. The statistical analyses were performed in GraphPad Prism 8 (GraphPad Software, USA). Representative experiments were repeated independently three times with similar results. *indicates P<0.05, **indicates P<0.01, ***indicates P<0.001, ****indicates P<0.0001, ns indicates P≥0.05.

### Example 3: IL2-ep13nsEV Prepared from DC were Educated with Tumor-Specific RNA

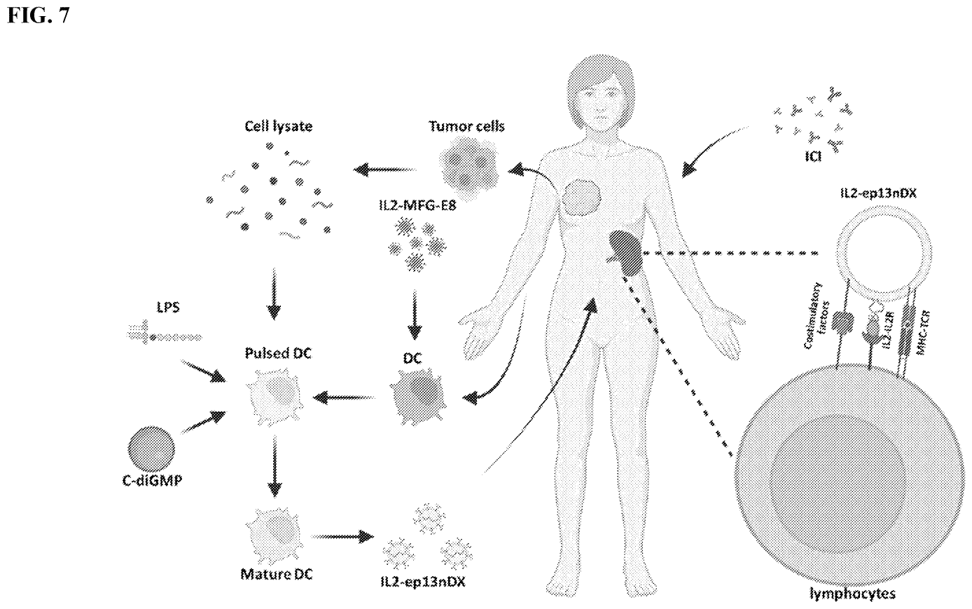

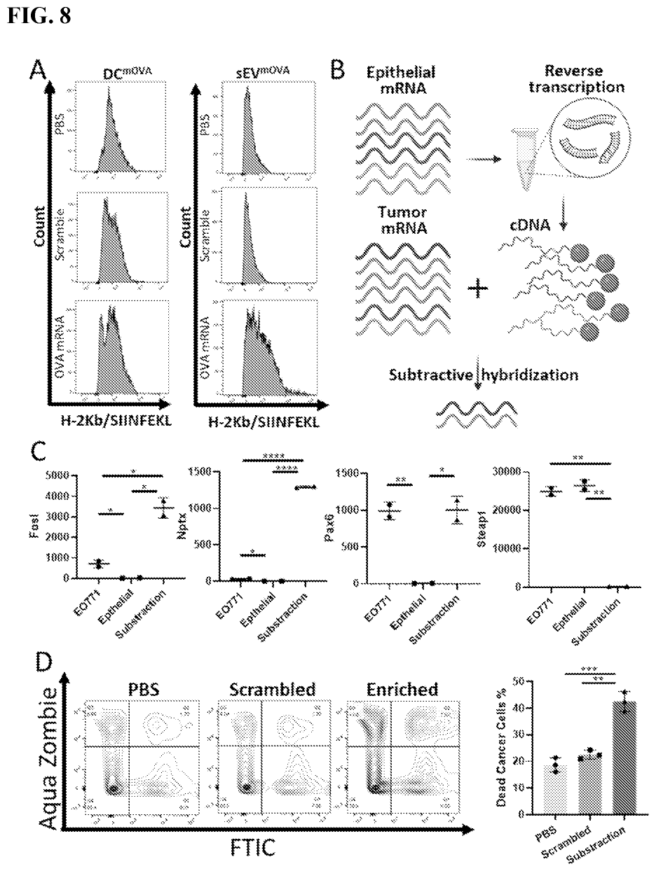

In Example 1, IL2-ep13nsEV prepared from DC were educated with tumor lysate and were shown to be able to effectively suppressed tumor growth. However, the use of tumor-specific RNAs for vaccination is considered to have multiple advantages over tumor cell lysates because they are easier to manipulate and amplify. It has been reported that mRNA vaccines can induce tumor-reactive lymphocytes against overexpressed wild-type antigens in cancer cells. Thus, we decided to explore the possibility of using IL2-ep13nsEV to express tumor-specific mRNAs and present these antigens to T cells. To test the feasibility first, OVA mRNA was used as a model system. The OVA mRNA was transfected into primary DC prepared from C57BL/6J mice and sEVs were prepared from transfected DC (DCSEV). We found both DC and DCSEV were able to present the OVA peptide on the surface bound to MHC I (FIG. 8 panel A), which indicates the feasibility of using DCSEV as antigen-presenting nanovesicles to present antigens of tumor-specific mRNAs. To separate tumor-specific mRNAs, we employed the mRNA subtractive hybridization approach (FIG. 8 panel B). The mRNA of normal breast epithelial cells was reverse transcribed into cDNA using Dynabeads Oligo (dT) 25. The mRNA of tumor cells was then hybridized with cDNA, followed by pull-down of the bound mRNA-cDNA-Dynabeads to isolate the tumor-specific mRNA. The selective enrichment of mRNAs that were overexpressed in tumor cells was verified by checking several highly expressed genes in EO771 cells. Fosl, Nptx, and Pax6 are known to be overexpressed by E0771 compared to normal epithelial cells, and subtractive hybridization resulted in significant enrichment of these three mRNAs after pull-down (FIG. 8 panel C). In contrast, Steap1 mRNA, which has similar levels in EO771 and epithelial cells, was mostly depleted by subtractive hybridization (FIG. 8 panel C). The optimized conditions of the buffer and temperature for hybridization were carefully examined, and SSPE buffer and hybridization at 0° C. were found to be the optimal conditions for enriching tumor-specific mRNAs (FIG. 8 panel D). Selective mRNAs enriched by subtractive hybridization were used to transfect DC, followed by the isolation of DCSEV. These DCSEVs were used to prime CD8⁺ T cells that were later used for co-culture with EO771 cells. DCsEVs generated using subtracted mRNAs were able to induce T cell cytotoxicity in EO771 cells (FIG. 8 panel E). Based on this finding, we decided to use the enriched mRNA from subtractive hybridization to construct DCsEV with engineered IL2 and enhanced levels of co-stimulatory factors following our previous protocol¹. This new antigen-presenting nanovesicle is named IL2/co-sEV^{mRNA}. With the mRNA collected from individual patient, this personalization (p13n) of IL2/co-sEV^{mRNA} leads to our study target.

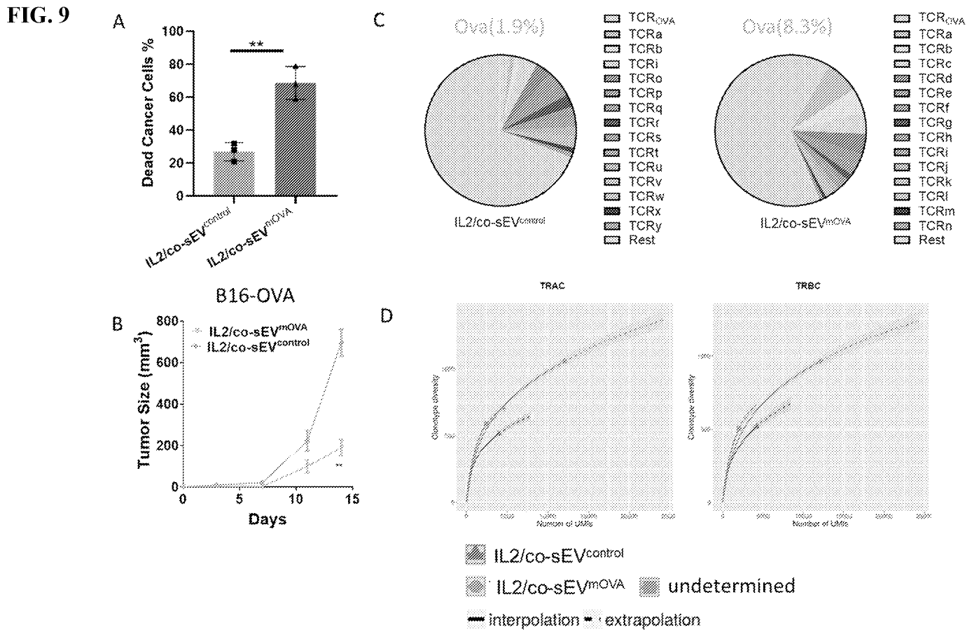

To test the feasibility of this approach, we performed the TCR sequencing using the OVA model with B16-OVA (MilliporeSigma) (FIG. 9). The IL2/co-sEV-based vaccine was generated by transfection of OVA mRNA or scrambled mRNA into engineered DCs, followed by IL2/co-sEV^{mOVA} purification. This vaccine was tested in vitro by T-cell killing assay (FIG. 9 panel A), which showed significant effect of IL2/co-sEV^{mOVA} vaccine. We then tested the efficacy in vivo using syngenetic mice with OVA-expressing tumors. We found that IL2/co-SEV^{mOVA} significantly suppressed the tumor growth (FIG. 9 panel B). To perform TCR sequencing, the tumors were collected after two weeks, and they were dissociated and CD3+ cells were isolated by FACS. The mRNA of the CD3+ cells were then collected and used to construct libraries using the QIAGEN QIAseq Immune Repertoire RNA Library Kit-T cell Receptor Panel (Qiagen). The libraries were sequenced using a MiSeq v3 (600 cycles) flow cell, targeting 25 million reads with a 2×300 paired-end read length. Data was analyzed using the GeneGlobe Data Analysis Center (Qiagen). We found that IL2/co-sEV^{mova} significantly expanded the T cell clonotypes infiltrating into the tumor and increased the frequency of OVA specific CD8⁺ T cells in the repertoire from 1.9% to 8.3% (FIG. 9 panels C and D). These results indicate that the IL2/co-SEV^{mova} vaccine induces mRNA-specific immune responses in tumors.

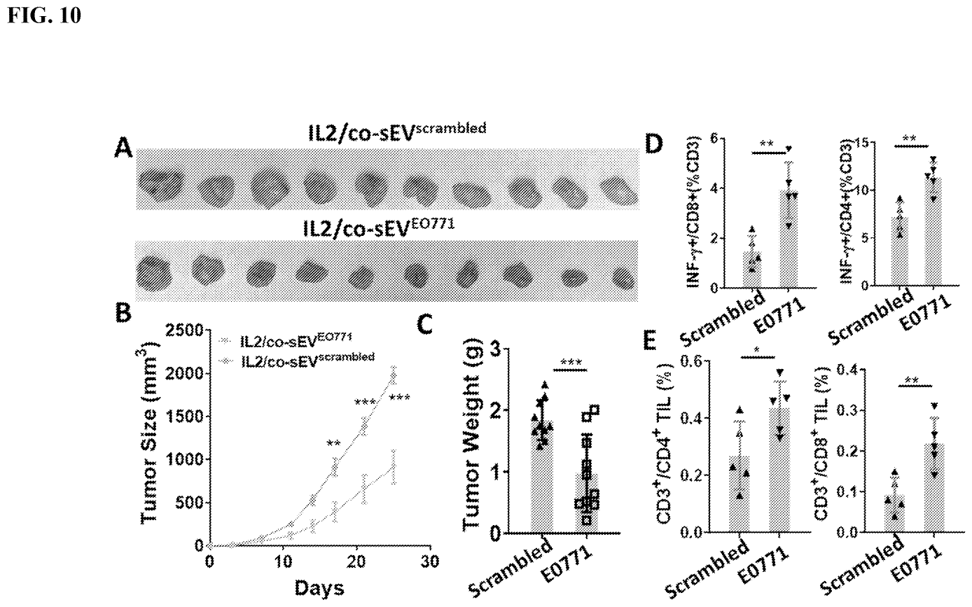

To further validate the treatment effect of IL2/co-sEV^{mRNA}, IL2/co-Sev^{Eo771} were prepared and tested in vivo by treating mice carrying EO771 tumors (FIG. 10 panel A). IL2/co-Sev^{Eo771} was found to significantly suppress the growth of EO771 primary tumors (FIG. 10 panels A-C) and increased the number of activated lymphocytes in the spleen (FIG. 10 panel D) as well as the CD4/CD8⁺ TIL in the tumors (FIG. 10 panel E).

The foregoing is illustrative of the present invention, and is not to be construed as limiting thereof. The invention is defined by the following claims, with equivalents of the claims to be included therein.

1. An isolated nonnucleated extracellular vesicle (EV) comprising a surface membrane and
a fusion protein comprising an interleukin-2 (IL-2) molecule and/or functional fragment thereof, a major histocompatibility (MHC) molecule and/or functional fragment thereof (e.g., MHC-I; e.g., MHC-II), and one or more costimulatory molecule, each expressed on the surface membrane of the EV;
wherein the MHC molecule and/or functional fragment thereof presents an exogenously introduced antigen.

2. The isolated EV of claim 1, wherein the exogenously introduced antigen is a cancer antigen (e.g., a tumor antigen, e.g., a cancer neoantigen).

3. The isolated EV of claim 1, wherein the EV is an exosome, an ectosome, a microvesicle, nanovesicle and/or an exosome-like vesicle.

4. The isolated EV of claim 1, wherein the one or more costimulatory molecule is 4-1BB, CD40L and/or Tim3.

5. The isolated EV of claim 1, wherein the EV is isolated from an antigen presenting cell (APC) of a mammalian subject (e.g., a healthy subject; e.g., a patient).

6. The isolated EV of claim 1, wherein the exogenously introduced antigen is isolated from a mammalian subject (e.g., a patient).

7. The isolated EV of claim 6, wherein the EV and the introduced antigen are isolated from the same mammalian subject (e.g., patient).

8. The isolated EV of claim 1, wherein the EV is about 50 nm in diameter to about 500 nm in diameter (e.g., about 50, 100, 150, 200, 250, 300, 350, 400, 450, or 500 nm in diameter or any value or range therein).

9. (canceled)

10. A composition comprising the isolated EV of claim 1, further comprising a pharmaceutically acceptable carrier, diluent, and/or adjuvant.

11. A method of producing an immune response to a disorder in a subject, comprising administering to the subject an effective amount of the isolated EV of claim 1.

12. A method of protecting a subject from the effects of a disorder, comprising administering to the subject an effective amount of the isolated EV of claim 1.

13. (canceled)

14. (canceled)

15. A method of treating a disorder in a subject, comprising administering to the subject (e.g., the subject having or suspected of having or developing the disorder) an effective amount of the isolated EV of claim 1.

16. (canceled)

17. (canceled)

18. A method of producing an immune response to a cancer in a subject, comprising administering to the subject an effective amount of the isolated EV of claim 1.

19. A method of preventing a disorder associated with a cancer in a subject, comprising administering to the subject an effective amount of the isolated EV of claim 1.

20. A method of protecting a subject from the effects of a cancer, comprising administering to the subject an effective amount of the isolated EV of claim 1.

21. (canceled)

22. (canceled)

23. A method of treating to a cancer in a subject, comprising administering to the subject an effective amount of the isolated EV of claim 1.

24-32. (canceled)

33. A method of producing a nonnucleated extracellular vesicle (EV) comprising a surface membrane and comprising an interleukin-2 (IL-2) molecule and/or functional fragment thereof, a major histocompatibility (MHC) molecule and/or functional fragment thereof, and one or more costimulatory molecule, each expressed on the surface membrane of the EV, wherein the MHC molecule and/or functional fragment thereof presents an exogenously introduced antigen, the method comprising:
(a) delivering to a culture of one or more antigen presenting cell that expresses major histocompatibility molecules I and II and which secretes nonnucleated EVs derived from the surface membrane of the APC (e.g., a macrophage, e.g., a dendritic cell), a IL-2 molecule and/or functional fragment thereof formulated to be expressed on the surface membrane of the APC, thereby producing an APC expressing the IL-2 molecule and/or functional fragment thereof on the surface membrane of the APC;
(b) contacting the APC of step (a) with one or more innate immune activator, thereby inducing expression of one or more costimulatory molecules on the surface membrane of the APC;
(c) introducing to the APC of step (b) a source of exogenous antigen (e.g., cancer cell lysate, e.g., isolated cancer cell RNA), thereby producing an APC expressing MHC-I and MHC-II, one or more costimulatory molecule and the IL-2 molecule and/or functional fragment thereof, on the surface membrane of the APC and on the surface membrane of the nonnucleated EVs secreted by the APC, wherein the MHC-I and/or MHC-II expressed on the surface membrane of the APC and the EVs presents the introduced exogenous antigen,
thereby producing a nonnucleated EV comprising a surface membrane and expressing MHC-I and MHC-II, one or more costimulatory molecule and the IL-2 molecule and/or functional fragment thereof, on the surface membrane of the EV, wherein the MHC-I and/or MHC-II expressed on the surface membrane of the EV presents the introduced exogenous antigen.

34. The method of claim 33, further comprising isolating the nonnucleated EVs secreted from the APC of step (c);
thereby producing the isolated nonnucleated extracellular vesicle comprising a surface membrane and expressing an interleukin-2 (IL-2) molecule and/or functional fragment thereof, a major histocompatibility (MHC) molecule and/or functional fragment thereof, and one or more costimulatory molecule, each expressed on the surface membrane of the EV, and
wherein the MHC molecule and/or functional fragment thereof presents an exogenously introduced antigen.

35. (canceled)

36. The method of claim 33, wherein the IL-2 molecule and/or functional fragment thereof comprises a fusion protein of an APC-surface molecule (e.g., MFG-E8 or the like) linked to an IL-2 functional fragment and wherein the innate immune activator molecule is a cGAS-STING cytosolic DNA sensing pathway activator and/or a Toll-like receptor (TLR) activator.

37-40. (canceled)

41. A method of treating a cancer in a subject in need thereof, comprising:
(a) retrieving a sample from the subject, wherein the sample comprises an APC of the subject that expresses major histocompatibility molecules I and II and which secretes nonnucleated EVs derived from the surface membrane of the APC (e.g., a macrophage, e.g., a dendritic cell);
(b) isolating one or more APC from the sample;
(c) delivering to a culture comprising one or more APC isolated from the sample, a IL-2 molecule and/or functional fragment thereof formulated to be expressed on the surface membrane of the APC;
(d) contacting-the APC of step (c) with one or more innate immune activator, thereby inducing expression of one or more costimulatory molecules on the surface membrane of the APC;
(e) introducing to the APC of step (d) a source of cancer antigen (e.g., cancer cell lysate, e.g., isolated cancer cell RNA) from the cancer of the subject, thereby producing an APC expressing MHC-I and MHC-II, one or more costimulatory molecule and the IL-2 molecule and/or functional fragment thereof, on the surface membrane of the APC and on the surface membrane of the EVs secreted by the APC, wherein the MHC-I and/or MHC-II expressed on the surface membrane of the APC and the EVs presents the introduced cancer antigen from the cancer of the subject,
(f) isolating the EVs secreted from the APC of step (e); and
(g) administering to the subject an effective amount of the isolated EVs and/or a composition comprising the same,
thereby treating the cancer in the subject in need thereof.
